(19) United States
(12) Patent Application Publication
(10) Pub. No.: US 2010/0081642 A1
(43) Pub. Date: Apr. 1, 2010

(54) CB-1 RECEPTOR MODULATOR PHARMACEUTICAL FORMULATIONS

(76) Inventors: Eleni Dokou, Harleysville, PA (US); Craig A. McKelvey, Ambler, PA (US); Kevin R. McKenney, Lansdale, PA (US); Andrey Peresypkin, Cambridge, MA (US); Karen C. Thompson, Lansdale, PA (US); W. Peter Wuelfing, Harleysville, PA (US)

Correspondence Address: MERCK AND CO., INC, P O BOX 2000, RAHWAY, NJ, 07065-0907, US

(21) Appl. No.: 12/558,977

(22) Filed: Sep. 14, 2009

Related U.S. Application Data
(60) Provisional application No. 61/100,479, filed on Sep. 26, 2008.

(51) Int. Cl. A61K 31/4245; A61P 3/04; A61P 25/28

(52) U.S. Cl. 514/210.2

(57) ABSTRACT

The present invention relates to pharmaceutical compositions that stabilize and improve the bioavailability of 3-[(1S)- 1-(1-{(S)-(4-chlorophenyl)[3-(5-oxo-4,5-dihydro-1,3,4-oxadiazol-2-yl)phenyl]methyl}azetidin-3-yl)-2-fluoro-2-methylpropyl]-5-fluorobenzonitrile. In particular, the present invention relates to pharmaceutical compositions that include 3-[(1S)-1-(1-{(S)-(4-chlorophenyl)[3 -(5-oxo-4,5-dihydro- 1,3,4-oxadiazol-2-yl)phenyl]methyl}azetidin-3-yl)-2-fluoro-2-methylpropyl]-5-fluorobenzonitrile and at least one pharmaceutical acceptable excipient, such as a mono-, di- or triglyceride.

### CROSS-REFERENCE TO RELATED APPLICATIONS

This application claims priority to and the benefit of U.S. Provisional Application Ser. No. 61/100,479, filed Sep. 26, 2008, the contents of which are hereby incorporated by reference.

### BACKGROUND

A major active ingredient in marijuana and hashish has been determined to be Δ9-tetrahydrocannabinol (Δ9-THC). Detailed research has revealed that the biological action of Δ9-THC and other members of the cannabinoid family occur through two G-protein coupled receptors termed Cannabinoid-1 (CB1) and Cannabinoid-2 (CB2). The CB1 receptor is primarily found in the central and peripheral nervous systems and to a lesser extent in several peripheral organs. Agonists of the CB1 receptor can cause sedation, hypothermia, intestinal immobility, antinociception, analgesia, catalepsy, anti-emesis, and appetite stimulation.

Conversely, antagonists and/or inverse agonists of the CB1 receptor can be useful in the treatment of psychosis, memory deficits, cognitive disorders, Alzheimer's disease, migraine, neuropathy, neuro-inflammatory disorders including multiple sclerosis and Guillain-Barre syndrome, the inflammatory sequelae of viral encephalitis, cerebral vascular accidents, head trauma, anxiety disorders, stress, epilepsy, Parkinson's disease, movement disorders, and schizophrenia. Such CB1 antagonists and/or inverse agonists can also be useful for the treatment of substance abuse disorders and eating disorders, particularly abuse and/or addiction to opiates, alcohol, marijuana, and nicotine, including smoking cessation and obesity. Certain CB1 inverse agonists such as, 3-[(1S)-1-(1-{(S)-(4-chlorophenyl)[3-(5-oxo-4,5-dihydro-1,3,4-oxadiazol-2-yl)phenyl]methyl}azetidin-3-yl)-2-fluoro-2-methylpropyl]-5-fluorobenzonitrile are described and disclosed in United States Published Application 2007/0123505, incorporated herein by reference in its entirety.

One drawback concerning certain CB1 antagonists/inverse agonists are that many of them are insoluble or have low solubility in water and are subject to degradation, which can pose challenges when it comes to formulating such CB1 antagonists/inverse agonist for administration. For example, 3-[(1S)-1-(1-{(S)-(4-chlorophenyl)[3-(5-oxo-4,5-dihydro-1,3,4-oxadiazol-2-yl)phenyl]methyl}azetidin-3-yl)-2-fluoro-2-methylpropyl]-5-fluorobenzonitrile is sensitive to oxidation. Thus, the present invention is directed to stable pharmaceutical compositions that include CB1 antagonist/inverse agonists such as 3-[(1S)-1-(1-{(S)-(4-chlorophenyl)[3-(5-oxo-4,5-dihydro-1,3,4-oxadiazol-2-yl)phenyl]methyl}azetidin-3-yl)-2-fluoro-2-methylpropyl]-5-fluorobenzonitrile, that limit the degradation and increase the bioavailability of the CB1 antagonist/inverse agonist.

### SUMMARY OF THE INVENTION

The pharmaceutical compositions described herein improve the bioavailability of 3-[(1S)-1-(1-{(S)-(4-chlorophenyl)[3-(5-oxo-4,5-dihydro-1,3,4-oxadiazol-2-yl)phenyl]methyl}azetidin-3-yl)-2-fluoro-2-methylpropyl]-5-fluorobenzonitrile. Additionally, the excipients included in the pharmaceutical compositions described herein provide chemical stability. Preferably, the pharmaceutical compositions described herein are administered orally in the form of capsules or tablets.

In certain embodiments, the excipients used in the capsules described herein can provide an adequate level of in vivo emulsification so that a large enough surface area is available for either 1) efficient transfer of 3-[(1S)-1-(1-{(S)-(4-chlorophenyl)[3-(5-oxo-4,5-dihydro-1,3,4-oxadiazol-2-yl)phenyl]methyl}azetidin-3-yl)-2-fluoro-2-methylpropyl]-5-fluorobenzonitrile from the pharmaceutical composition to GI fluids, or 2) efficient absorption of 3-[(1S)-1-(1-{(S)-(4-chlorophenyl)[3-(5-oxo-4,5-dihydro-1,3,4-oxadiazol-2-yl)phenyl]methyl}azetidin-3-yl)-2-fluoro-2-methylpropyl]-5-fluorobenzonitrile in the GI tract. In such embodiments, the pharmaceutical compositions used to fill the capsule, when dissolved in aqueous medium, may create a colloidal media consisting of micelles, emulsion, microemulsion or other colloidal structures, which may dissolve 3-[(S)-1-(1-{(S)-(4-chlorophenyl)[3-(5-oxo-4,5-dihydro-1,3,4-oxadiazol-2-yl)phenyl]methyl}azetidin-3-yl)-2-fluoro-2-methylpropyl]-5-fluorobenzonitrile thus, increasing the dissolution rate of 3-[(S)-1-(1-{(S)-(4-chlorophenyl)[3-(5-oxo-4,5-dihydro-1,3,4-oxadiazol-2-yl)phenyl]methyl}azetidin-3-yl)-2-fluoro-2-methylpropyl]-5-fluorobenzonitrile.

In certain embodiments described herein, the capsules include 3-[(1S)-1-(1-{(S)-(4-chlorophenyl)[3-(5-oxo-4,5-dihydro-1,3,4-oxadiazol-2-yl)phenyl]methyl}azetidin-3-yl)-2-fluoro-2-methylpropyl]-5-fluorobenzonitrile and at least one pharmaceutical acceptable excipient, such as a mono-, di- or triglyceride. For example, the pharmaceutical compositions include 3-[(1S)-1-(1-{(S)-(4-chlorophenyl)[3-(5-oxo-4,5-dihydro-1,3,4-oxadiazol-2-yl)phenyl]methyl}azetidin-3-yl)-2-fluoro-2-methylpropyl]-5-fluorobenzonitrile and a triglyceride, such as fractionated coconut oils such as known and commercially available under the trade name MIGLYOL, including the products: MIGLYOL 810; MIGLYOL 812; MIGLYOL 818; and CAPTEX 355.

In still other embodiments described herein, the capsules include 3-[(1S)-1-(1-{(S)-(4-chlorophenyl)[3-(5-oxo-4,5-dihydro-1,3,4-oxadiazol-2-yl)phenyl]methyl}azetidin-3-yl)-2-fluoro-2-methylpropyl]-5-fluorobenzonitrile; a triglyceride and an antioxidant. For example, the pharmaceutical compositions include 3-[(1S)-1-(1-{(S)-(4-chlorophenyl)[3-(5-oxo-4,5-dihydro-1,3,4-oxadiazol-2-yl)phenyl]methyl}azetidin-3-yl)-2-fluoro-2-methylpropyl]-5-fluorobenzonitrile and a triglyceride, such as fractionated coconut oils such as known and commercially available under the trade name MIGLYOL, including the products: MIGLYOL 810; MIGLYOL 812; MIGLYOL 818; and CAPTEX 355 and butylated hydroxyaniole (BHA).

In yet other embodiments described herein, the capsules include 3-[(1S)-1-(1-{(S)-(4-chlorophenyl)[3-(5-oxo-4,5-dihydro-1,3,4-oxadiazol-2-yl)phenyl]methyl}azetidin-3-yl)-2-fluoro-2-methylpropyl]-5-fluorobenzonitrile; at least one surfactant and an antioxidant. For example, the pharmaceutical compositions include 3-[(1S)-1-(1-{(S)-(4-chlorophenyl)[3-(5-oxo-4,5-dihydro-1,3,4-oxadiazol-2-yl)phenyl]methyl}azetidin-3-yl)-2-fluoro-2-methylpropyl]-5-fluorobenzonitrile and caprylic/capric glycerides such as known and commercially available under the trade name IMWITOR 742; polysorbate such as known and commercially available under the trade name TWEEN 80 and buylated hydroxyaniole (BHA).

Other embodiments described herein, include pharmaceutical compositions in the form of tablets. In certain embodiments, the excipients used in the tablets described herein stabilize 3-[(1S)-1-(1-{(S)-(4-chlorophenyl)[3-(5-oxo-4,5-dihydro-1,3,4-oxadiazol-2-yl)phenyl]methyl}azetidin-3-yl)-2-fluoro-2-methylpropyl]-5-fluorobenzonitrile. In certain embodiments, the tablets described herein include an effective and uniform distribution of an antioxidant which prevents 3-[(1S)-1-(1-{(S)-(4-chlorophenyl)[3-(5-oxo-4,5-dihydro-1,3,4-oxadiazol-2-yl)phenyl]methyl}azetidin-3-yl)-2-fluoro-2-methylpropyl]-5-fluorobenzonitrile from oxidizing.

In certain embodiments described herein, the tablets include 3-[(1S)-1-(1-{(S)-(4-chlorophenyl)[3-(5-oxo-4,5-dihydro-1,3,4-oxadiazol-2-yl)phenyl]methyl}azetidin-3-yl)-2-fluoro-2-methylpropyl]-5-fluorobenzonitrile and at least one pharmaceutical acceptable excipient, such as an antioxidant. For example, the pharmaceutical compositions include 3-[(1S)-1-(1-{(S)-(4-chlorophenyl)[3-(5-oxo-4,5-dihydro-1,3,4-oxadiazol-2-yl)phenyl]methyl}azetidin-3-yl)-2-fluoro-2-methylpropyl]-5-fluorobenzonitrile, butylated hydroxyaniole (BHA) and at least one additional excipient such as, microcrystalline cellulose, croscarmellose, lactose monohydrate, hydroxypropyl cellulose and magnesium stearate.

### DETAILED DESCRIPTION OF THE INVENTION

### Definitions

The term “diabetes,” as used herein, includes both insulin-dependent diabetes mellitus (i.e., IDDM, also known as Type I diabetes) and non-insulin-dependent diabetes mellitus (i.e., NIDDM, also known as Type II diabetes). Type I diabetes, or insulin-dependent diabetes, is the result of an absolute deficiency of insulin, the hormone which regulates glucose utilization. Type II diabetes, or insulin-independent diabetes (i.e., non-insulin-dependent diabetes mellitus), often occurs in the face of normal, or even elevated levels of insulin and appears to be the result of the inability of tissues to respond appropriately to insulin. Most of the Type II diabetics are also obese. The compositions of the present invention are useful for treating both Type I and Type II diabetes. The compositions are especially effective for treating Type II diabetes. The compositions of the present invention are also useful for treating and/or preventing gestational diabetes mellitus.

As used herein, the term “substance abuse disorders” includes substance dependence or abuse with or without physiological dependence. The substances associated with these disorders are: alcohol, amphetamines (or amphetamine-like substances), caffeine, cannabis, cocaine, hallucinogens, inhalants, marijuana, nicotine, opioids, phencyclidine (or phencyclidine-like compounds), sedative-hypnotics or benzodiazepines, and other (or unknown) substances and combinations of all of the above.

In particular, the term “substance abuse disorders” includes drug withdrawal disorders such as alcohol withdrawal with or without perceptual disturbances; alcohol withdrawal delirium; amphetamine withdrawal; cocaine withdrawal; nicotine withdrawal; opioid withdrawal; sedative, hypnotic or anxiolytic withdrawal with or without perceptual disturbances; sedative, hypnotic or anxiolytic withdrawal delirium; and withdrawal symptoms due to other substances. It will be appreciated that reference to treatment of nicotine withdrawal includes the treatment of symptoms associated with smoking cessation.

Other “substance abuse disorders” include substance-induced anxiety disorder with onset during withdrawal; substance-induced mood disorder with onset during withdrawal; and substance-induced sleep disorder with onset during withdrawal.

As used herein the term “obesity” refers to a condition in which there is an excess of body fat. The operational definition of obesity is based on the Body Mass Index (BMI), which is calculated as body weight per height in meters squared (kg/m2). “Obesity” refers to a condition whereby an otherwise healthy subject has a BMI greater than or equal to 30 kg/m2, or a condition whereby a subject with at least one co-morbidity has a BMI greater than or equal to 27 kg/m2. An “obese subject” is an otherwise healthy subject with a BMI greater than or equal to 30 kg/m2 or a subject with at least one co-morbidity with a BMI greater than or equal to 27 kg/m2. A “subject at risk for obesity” is an otherwise healthy subject with a BMI of 25 kg/m2 to less than 30 kg/m2 or a subject with at least one co-morbidity with a BMI of 25 kg/m2 to less than 27 kg/m2.

The increased risks associated with obesity occur at a lower BMI in Asians. In Asian countries, including Japan, “obesity” refers to a condition whereby a subject with at least one obesity-induced or obesity-related co-morbidity that requires weight reduction or that would be improved by weight reduction, has a BMI greater than or equal to 25 kg/m2. In Asian countries, including Japan, an “obese subject” refers to a subject with at least one obesity-induced or obesity-related co-morbidity that requires weight reduction or that would be improved by weight reduction, with a BMI greater than or equal to 25 kg/m2. In Asian countries, a “subject at risk of obesity” is a subject with a BMI of greater than 23 kg/m2 to less than 25 kg/m2.

The terms “prevent(s)”, “prevented”, “preventing” or “prevention” are used herein interchangeably and refer to any prevention or any contribution to the prevention of a condition in an animal or the development of a condition if none has occurred in an animal which may be predisposed to such condition but has not yet been inflicted with or diagnosed as having such condition.

As used herein the terms “subject(s)”, “individual(s)” and “patient(s)” can be used interchangeably and refer to a mammal, preferably a human, who has been the object of treatment, observation or experiment. In one embodiment the term “mammal” is a “human” said human being either male or female. The instant combinations are also useful for treating or preventing obesity and obesity-related conditions in cats and dogs. As such, the term “mammal” includes companion animals such as cats and dogs.

As used herein the phrase “subject in need thereof” refers to a subject who is in need of treatment or prophylaxis as determined by a researcher, veterinarian, medical doctor or other clinician. In one embodiment, a subject in need thereof is a mammal. In another embodiment, a subject in need thereof is a human. In another yet embodiment, a subject in need thereof is an obese subject. In another embodiment, a subject in need thereof is an obese subject with diabetes. In another embodiment, a subject in need thereof is an obese subject at risk of developing diabetes. In another embodiment, a subject in need thereof is an obese diabetic subject. In another embodiment, a subject in need thereof is a diabetic subject at risk of developing obesity. In another embodiment, a subject in need thereof is a subject suffering from Alzheimer's disease. In another embodiment, a subject in need thereof is a subject at risk of developing Alzheimer's disease.

As used herein the phrase “pharmaceutically acceptable salt” refers to salts prepared from pharmaceutically acceptable non-toxic bases or acids including inorganic or organic bases and inorganic or organic acids. Salts of basic compounds encompassed within the term “pharmaceutically acceptable salt” refer to non-toxic salts of the compounds of this invention which are generally prepared by reacting the free base with a suitable organic or inorganic acid. Representative salts of basic compounds of the present invention include, but are not limited to, the following: acetate, benzenesulfonate, benzoate, bicarbonate, bisulfate, bitartrate, borate, bromide, camsylate, carbonate, chloride, clavulanate, citrate, edetate, edisylate, estolate, esylate, fumarate, gluceptate, gluconate, glutamate, hexylresorcinate, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, oleate, oxalate, pamoate (embonate), palmitate, pantothenate, phosphate/diphosphate, polygalacturonate, salicylate, stearate, sulfate, subacetate, succinate, tannate, tartrate, teoclate, tosylate, triethiodide and valerate. Furthermore, where the compounds of the invention carry an acidic moiety, suitable pharmaceutically acceptable salts thereof include, but are not limited to, salts derived from inorganic bases including aluminum, ammonium, calcium, copper, ferric, ferrous, lithium, magnesium, manganic, mangamous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, cyclic amines, and basic ion-exchange resins, such as arginine, betaine, caffeine, choline, N,N-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, isopropylamine, lysine, methylglucamine, morpholine, piperazine, piperidine, polyamine resins, procaine, purines, theobromine, triethylamine, trimethylamine, tripropylamine, tromethamine, and the like.

The pharmaceutically acceptable salts of the pharmaceutical formulations described herein include formulations wherein one of the individual components of the formulation is in the form of a pharmaceutically acceptable salt, or formulations wherein some or all of the individual components are in the form of pharmaceutically acceptable salts (wherein the salts for each of the components can be the same or different), or a pharmaceutically acceptable salt of the combined components (i.e., a salt of the composition).

The terms “treat(s)”, “treated”, “treating” or “treatment” are used herein interchangeably and refer to any treatment of a condition in an animal, such as a human, diagnosed or inflicted with such condition and includes, but is not limited to: (a) caring for an animal diagnosed or inflicted with a condition; (b) curing or healing an animal diagnosed or inflicted with a condition; (c) causing regression of a condition in an animal; (d) arresting further development or progression of a condition in an animal; (e) slowing the course of a condition in an animal; (f) relieving, improving, decreasing or stopping symptoms a condition in a animal; (g) relieving, decreasing or stopping the symptoms caused by or associated with a condition in an animal; or (h) reducing the frequency, number or severity of episodes caused by or associated with a condition in an animal.

As used herein the term “surfactant” means any surface acting agent.

### CB1 Inverse Agonist

Described herein are stable pharmaceutical compositions that increase the bioavailability of certain CB1 inverse agonists. In certain embodiments, described herein are stable pharmaceutical compositions of 3-[(1S)-1-(1-{(S)-(4-chlorophenyl)[3-(5-oxo-4,5-dihydro-1,3,4-oxadiazol-2-yl)phenyl]methyl}azetidin-3-yl)-2-fluoro-2-methylpropyl]-5-fluorobenzonitrile that increase the bioavailability of 3-[(1S)-1-(1-{(S)-(4-chlorophenyl)[3-(5-oxo-4,5-dihydro-1,3,4-oxadiazol-2-yl)phenyl]methyl}azetidin-3-yl)-2-fluoro-2-methylpropyl]-5-fluorobenzonitrile as compared to 3-[(1S)-1-(1-{(S)-(4-chlorophenyl)[3-(5-oxo-4,5-dihydro-1,3,4-oxadiazol-2-yl)phenyl]methyl}azetidin-3-yl)-2-fluoro-2-methylpropyl]-5-fluorobenzonitrile in dry-filled capsules. The structural formula of 3-[(1S)-1-(1- (S)-(4-chlorophenyl)[3-(5-oxo-4,5-dihydro-1,3,4-oxadiazol-2-yl)phenyl]methyl}azetidin-3-yl)-2-fluoro-2-methylpropyl]-5-fluorobenzonitrile is below:

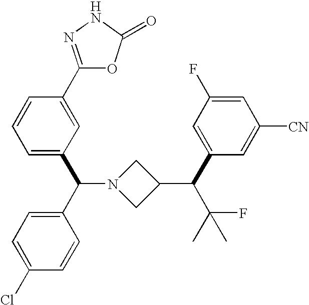

In certain embodiments, 3-[(1S)-1-(1-{(S)-(4-chlorophenyl)[3-(5-oxo-4,5-dihydro-1,3,4-oxadiazol-2-yl)phenyl]methyl}azetidin-3-yl)-2-fluoro-2-methylpropyl]-5-fluorobenzonitrile, is unsolvated.

In another embodiment, the 3-[(S)-1-(1-{(S)-(4-chlorophenyl)[3-(5-oxo-4,5-dihydro-1,3,4-oxadiazol-2-yl)phenyl]methyl}azetidin-3-yl)-2-fluoro-2-methylpropyl]-5-fluorobenzonitrile, is a solvate or a hemisolvate.

In yet another embodiment, the 3-[(S)-1-(1-{(S)-(4-chlorophenyl)[3-(5-oxo-4,5-dihydro-1,3,4-oxadiazol-2-yl)phenyl]methyl}azetidin-3-yl)-2-fluoro-2-methylpropyl]-5-fluorobenzonitrile, is the unsolvated free base.

In still another embodiment, the 3-[(S)-1-(1-{(S)-(4-chlorophenyl)[3-(5-oxo-4,5-dihydro-1,3,4-oxadiazol-2-yl)phenyl]methyl}azetidin-3-yl)-2-fluoro-2-methylpropyl]-5-fluorobenzonitrile, is the unsolvated salt.

In still another class of this embodiment, the 3-[(1S)-1-(1-{(S)-(4-chlorophenyl)[3-(5-oxo-4,5-dihydro-1,3,4-oxadiazol-2-yl)phenyl]methyl}azetidin-3-yl)-2-fluoro-2-methylpropyl]-5-fluorobenzonitrile, is the unsolvated HCl or methyl sulfonic acid (MSA) salt.

In another class of this embodiment, the 3-[(1S)-1-(1-{(S)-(4-chlorophenyl)[3-(5-oxo-4,5-dihydro-1,3,4-oxadiazol-2-yl)phenyl]methyl}azetidin-3-yl)-2-fluoro-2-methylpropyl]-5-fluorobenzonitrile, is the solvated HCl or MSA salt.

Methods of making 3-[(1S)-1-(1-{(S)-(4-chlorophenyl)[3-(5-oxo-4,5-dihydro-1,3,4-oxadiazol-2-yl)phenyl]methyl}azetidin-3-yl)-2-fluoro-2-methylpropyl]-5-flurobenzonitrile are described in United States Published Application 2007/0123505, incorporated herein by reference in its entirety.

3-[(1S)-1-(1-{(S)-(4-chlorophenyl)[3-(5-oxo-4,5-dihydro-1,3,4-oxadiazol-2-yl)phenyl]methyl}azetidin-3-yl)-2-fluoro-2-methylpropyl]-5-fluorobenzonitrile exhibits poor solubility in water, 0.001 mg/ml in water. However, the bioavailability of 3-[(1S)-1-(1-{(S)-(4-chlorophenyl)[3-(5-oxo-4,5-dihydro-1,3,4-oxadiazol-2-yl)phenyl]methyl}azetidin-3-yl)-2-fluoro-2-methylpropyl]-5-fluorobenzonitrile is increased when 3-[(1S)-1-(1- (S)-(4-chlorophenyl)[3-(5-oxo-4,5-dihydro-1,3,4-oxadiazol-2-yl)phenyl]methyl}azetidin-3-yl)-2-fluoro-2-methylpropyl]-5-fluorobenzonitrile is formulated into a pharmaceutical composition that includes certain liquid, semi-solid or solid excipients.

The pharmaceutical compositions described herein can include 0.01% to 25% by weight of 3-[(1S)-1-(1-{(S)-(4-chlorophenyl)[3-(5-oxo-4,5-dihydro-1,3,4-oxadiazol-2-yl)phenyl]methyl}azetidin-3-yl)-2-fluoro-2-methylpropyl]-5-fluorobenzonitrile. In certain embodiments described herein the pharmaceutical compositions described herein can include 0.01% to 0.1%; 0.1% to 1%; 1% to5%; 5% to 10%; or 10% to 25% by weight of 3-[(1S)-1-(1-{(S)-(4-chlorophenyl)[3-(5-oxo-4,5-dihydro-1,3,4-oxadiazol-2-yl)phenyl]methyl}azetin-3-yl)-2-fluoro-2-methylpropyl]-5-fluorobenzonitrile free base.

For example the pharmaceutical compositions described herein can include, about or exactly, 0.1%, 0.2%, 0.3%, 0.4%, 0.5%, 0.6%, 0.7%, 0.8%, 0.9%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, or 10% by weight of 3-[(1S)-1-(1-{(S)-(4-chlorophenyl)[3-(5-oxo-4,5-dihydro-1,3,4-oxadiazol-2-yl)phenyl]methyl}azetidin-3-yl)-2-fluoro-2-methylpropyl]-5-fluorobenzonitrile.

### Compositions

The pharmaceutical compositions described herein include 3-[(1S)-1-(1-(S)-(4-chlorophenyl)[3-(5-oxo-4,5-dihydro-1,3,4-oxadiazol-2-yl)phenyl]methyl}azetidin-3-yl)-2-fluoro-2-methylpropyl]-5-fluorobenzonitrile and at least one pharmaceutically acceptable excipient.

In certain embodiments, the pharmaceutically acceptable excipients are mono-, di, and/or triglycerides. Mono-, di- and triglycerides that may be in the pharmaceutical compositions described herein, include those that are derived from C₆, C₈, C₁₀, C₁₂, C₁₄, C₁₆, C₁₈, C₂₀ and C₂₂ fatty acids.

Sources of suitable mono-, di- and triglycerides include animal (cow- or hog-derived) and vegetable sources. Synthetic mono-, di- and triglycerides may also be suitable. Suitable examples of monoglycerides include, but are not limited to, 2-arachidonoylglycerol, glyceryl monostearate, glycerylmonopalmitate, glycerylmonolaurate, glycerylmonooleate, glycerylmonoerucate and glycerylmono ricinoleate.

Suitable examples of diglycerides include, but are not limited to, diolein, dipalmitolein, and mixed caprylin-caprin diglycerides.

Suitable examples of triglycerides include, but are not limited to, vegetable oils, fish oils, animal fats, hydrogenated vegetable oils, partially hydrogenated vegetable oils, synthetic triglycerides, modified triglycerides, fractionated triglycerides, medium and long-chain triglycerides, structured triglycerides, and mixtures thereof.

More specific examples of suitable triglycerides include, but are not limited to, almond oil; babassu oil; borage oil; blackcurrant seed oil; canola oil; castor oil; coconut oil; corn oil; cottonseed oil; evening primrose oil; grapeseed oil; groundnut oil; mustard seed oil; olive oil; palm oil; palm kernel oil; peanut oil; rapeseed oil; safflower oil; sesame oil; shark liver oil; soybean oil; sunflower oil; hydrogenated castor oil; hydrogenated coconut oil; hydrogenated palm oil; hydrogenated soybean oil; hydrogenated vegetable oil; hydrogenated cottonseed and castor oil; partially hydrogenated soybean oil; partially hydrogenated soy; partially hydrogenated cottonseed oil; glyceryl tricaproate; glyceryl tricaprylate; glyceryl tricaprate; glyceryl triundecanoate; glyceryl trilaurate; glyceryl trioleate; glyceryl trilinoleate; glyceryl trilinolenate; glyceryl tricaprylate/caprate; glyceryl tricaprylate/caprate/laurate; glyceryl tricaprylate/caprate/linoleate; and glyceryl tricaprylate/caprate/stearate.

Additional suitable triglycerides include, but are not limited to, the medium chain triglyceride available under the trade name LABRAFAC CC, neutral oils, e.g. neutral plant oils, in particular fractionated coconut oils such as known and commercially available under the trade name MIGLYOL, including the products: MIGLYOL 810; MIGLYOL 812; MIGLYOL 818; and CAPTEX 355.

Also suitable are caprylic-capric acid triglycerides such as known and commercially available under the trade name MYRITOL, including the product MYRITOL 813. Further suitable products of this class are CAPMUL MCT, CAPTEX 200, CAPTEX 300, CAPTEX 800, NEOBEE M5 and MAZOL 1400.

It is understood by those of ordinary skill in the art that several commercial surfactant compositions contain small to moderate amounts of triglycerides, typically as a result of incomplete reaction of a triglyceride starting material in, for example, a trans-esterification reaction. Thus, the surfactants that contain a triglyceride are suitable for use in the pharmaceutical compositions described herein. Examples of commercial surfactant compositions containing triglycerides include some members of the surfactant families GELUCIRES, MAISINES, AND IMWITORS. Specific examples of these compounds are GELUCIRE 44/14 (saturated polyglycolized glycerides); GELUCIRE 50/13 (saturated polyglycolized glycerides); GELUCIRE 53/10 (saturated polyglycolized glycerides); GELUCIRE 33/01 (semi-synthetic triglycerides of C₈-C₁₈ saturated fatty acids); GELUCIRE 39/01 (semi-synthetic glycerides); other GELUCIRE, such as 37/06, 43/01, 35/10, 37/02, 46/07, 48/09, 50/02, 62/05, etc.; MAISINE 35-1 (linoleic glycerides); and IMWITOR 742 (caprylic/capric glycerides).

In certain embodiments the pharmaceutical compositions described herein include, 3-[(1S)-1-(1-{(S)-(4-chlorophenyl)[3-(5-oxo-4,5-dihydro-1,3,4-oxadiazol-2-yl)phenyl]methyl}azetidin-3-yl)-2-fluoro-2-methylpropyl]-5-fluorobenzonitrile, caprylic/capric glycerides, such as IMWITOR 742 and/or fractionated coconut oils such as known and commercially available under the trade name MIGLYOL, including the products: MIGLYOL 810; MIGLYOL 812; MIGLYOL 818; and CAPTEX 355.

The pharmaceutical compositions described here in can contain between 75% to 99.9% by weight of a mono-, di, triglyceride or between 75% to 99.9% by weight of a surfactant or commercial product that includes a suitable mono-, di, triglyceride. In certain embodiments, the pharmaceutical compositions described herein can include, 75% to 85%, 85% to 90%, 90% to 95%, 95% to 99%, or 99% to 99.9% by weight of a mono-, di, triglyceride or between 75% to 85%, 85% to 90%, 90% to 95%, 95% to 99%, or 99% to 99.9% by weight of a surfactant or commercial product that includes a suitable mono-, di, triglyceride. For example, the pharmaceutical compositions described herein can include (about or exactly) 75%, 76%, 77%, 78%, 79%, 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, or 99.9% by weight of a suitable mono-, di, triglyceride or (about or exactly) 75%, 76%, 77%, 78%, 79%, 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, or 99.9% by weight of a surfactant or commercial product that includes a suitable mono-, di, triglyceride.

In still other embodiments described herein, pharmaceutical compositions described herein include an antioxidant. Suitable antioxidants include, but are not limited to, Vitamin E, tocopherols, tocotrienols, phenols, BHA, BHT, thiols, sulfides, disulfides, sulfoxides, hydroquinones, ascorbyl palmitate, phenylenediamines, thiocarbamates, gallates, and any combination thereof. The pharmaceutical compositions described herein can include 0.001% to 1% by weight of an antioxidant. In certain embodiment the pharmaceutical compositions described herein can include (about or exactly) 0.001%, 0.002%, 0.003%, 0.004%, 0.005%, 0.006%, 0.007%, 0.008%, 0.009%, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9% or 1% by weight of an antioxidant.

Additional excipients that can be included in the pharmaceutical compositions described herein can also include surfactants, diluents, binders, vehicles, carriers, disintegrating agents, lubricants, swelling agents, solubilizing agents, wicking agents, cooling agents, preservatives, stabilizers, sweeteners and flavorings.

For example, the pharmaceutical compositions described herein can further include at least one surfactant that does not include a triglyceride. Suitable surfactants include cationic, anionic, and nonionic surfactants. These include, but are not limited to, for example fatty acids and alkyl sulfonates; cationic surfactants such as benzalkonium chloride, examples of which include Hyamine 1622; anionic surfactants, such as dioctyl sodium sulfosuccinate, examples of which include Docusate Sodium and sodium lauryl sulfate, examples of which include sodium dodecyl sulfate; sorbitan fatty acid esters, examples of which include the SPAN series of surfactants; Vitamin E TPGS; polyoxyethylene sorbitan fatty acid esters, examples of which include the TWEEN series of surfactants; polyoxyethylene castor oils and hydrogenated castor oils such as CREMPHOR RH-40 and CREMPHOR EL; LIPOSORB P-20; CAPMUL POE-0; poloxamers, such as poloxamer 407 and natural surfactants such as sodium taurocholic acid, 1-palmitoyl-2-oleoyl-sn-glycero-3-phosphocholine, lecithin, and other phospholipids When any of the surfactants that do not contain triglycerides are used in conjunction with mono-, di-, triglycerides or any surfactant or commercial product that contains mono-, di-, triglycerides, such excipients can be combined in a ratio of 10:1 to 1:10 (surfactants that do not contain triglycerides: mono-, di-, triglycerides or any surfactant or commercial product that contains mono-, di-, triglycerides). In certain embodiments, the pharmaceutical compositions described herein may contain a mixture of at least one surfactant that does not contain triglycerides and a mono-, di-, triglycerides or a surfactant or commercial product that contains mono-, di-, triglycerides in a ratio or 2:1, 1:1 or 1:2 (surfactants that do not contain triglycerides : mono-, di-, triglycerides or any surfactant or commercial product that contains mono-, di-, triglycerides).

For example, in one embodiment the pharmaceutical compositions described herein include 3-[(1S)-1-(1-{(S)-(4-chlorophenyl)[3-(5-oxo-4,5-dihydro-1,3,4-oxadiazol-2-yl)phenyl]methyl}azetidin-3-yl)-2-fluoro-2-methylpropyl]-5-fluorobenzonitrile, caprylic/capric glycerides such as IMWITOR 742 and Polysorbitan 80 such as TWEEN 80 in a 1:1 ratio, and, optionally, an antioxidant.

Another embodiment of the pharmaceutical compositions described herein include 0.8% to 2.4% 3-[(1S)-1-(1-{(S)-(4-chlorophenyl)[3-(5-oxo-4,5-dihydro-1,3,4-oxadiazol-2-yl)phenyl]methyl}azetidin-3-yl)-2-fluoro-2-methylpropyl]-5-fluorobenzonitrile by weight, 48.7% to 49.6% Polysorbate 80 by weight, 48.7% to 49.6% IMWITOR 742 by weight, and 0.06% butylated hydroxyanisole by weight.

Examples of stabilizers or preservatives include, for example, parahydroxybenzoic acid alkyl esters, antioxidants, antifungal agents, and other stabilizers/preservatives known in the art.

Examples of coloring agents include, for example, water soluble dye, Lake dye, iron oxide, natural colors, titanium oxide, and the like.

Examples of diluents or fillers include water-soluble and/or water-insoluble fillers. The water-soluble diluent agent may be constituted from a polyol of less than 13 carbon atoms, in the form of directly compressible material (the mean particle size being between about 100 and about 500 microns), in the form of a powder (the mean particle size being less than about 100 microns) or a mixture thereof. The polyol is preferably chosen from the group comprising of mannitol, xylitol, sorbitol and maltitol. The water-insoluble diluent agent may be a cellulosic derivative preferably microcrystalline cellulose. Especially preferred diluents are those with minimal moisture content, such as lactose monohydrate and magnesium oxide.

Examples of disintegrating agents include, but are not limited to, crosslinked sodium carboxymethylcellulose, crospovidone and their mixtures. A part of the disintegrating agent may be used for the preparation of PPI, cholinergic agonist, parietal activator and/or antacid granules.

Examples of lubricating agents include, but are not limited to, magnesium stearate, stearic acid and its pharmaceutically acceptable alkali metal salts, sodium stearylfumarate, Macrogol 6000, glyceryl behenate, talc, colloidal silicon dioxide, calcium stearate, sodium stearate, Cab-O-Sil, Syloid, sodium lauryl sulfate, sodium chloride, magnesium lauryl sulfate, talc and their mixtures. A portion of the lubricant may be used as an internal solid lubricant which is blended and granulated with other components of the granulation. Another portion of the lubricant may be added into the final blended material just before compression or encapsulation that coats the outside of the granules in the final blend.

Examples of swelling agents include, but are not limited to, starches; polymers; cellulosic materials, such as, microcrystalline cellulose, hydroxypropylmethyl cellulose, sodium carboxymethylcellulose and ethyl cellulose; waxes such as bees wax; natural materials, such as, gums and gelatins; or mixtures of any of the above.

Examples of binders include, but are not limited to, gum tragacanth (arabic), acacia, corn starch, potato starch, alginic acid, povidone, acacia, alginic acid, ethylcellulose, methylcellulose, microcrystalline cellulose, a derivatized cellulose, such as carboxymethyl cellulose, sodium carboxymethyl cellulose, hydroxyethyl cellulose, hydroxypropyl methylcellulose, and hydroxypropyl cellulose, dextrin, gelatin, glucose, guar gum, hydrogenated vegetable oil, type I, polyethylene glycol, lactose, lactose monohydrate, compressible sugars, sorbitol, mannitol, dicalcium phosphate dihydrate, tricalcium phosphate, calcium sulfate dihydrate, maltodextrins, lactitol, magnesium carbonate, xylitol, magnesium aluminium silicate, maltodextrin, methylcellulose, hydroxypropylcellulose, polyethylene, polyethylene oxide, polymethacrylates, plasdone, sodium alginate, starch, pregelatinized starch, zein or the like; a sweetening agent such as sucrose, potassium acesulfame, aspartame, lactose, dihydrochalcone neohesperidine, saccharin, sucralose, polyols such as xylitol, mannitol, and maltitol, sodium saccharide, Asulfame-K, NEOTAME®, glycyrrhizin, malt syrup and combinations thereof; a flavoring such as berry, orange, peppermint, oil of wintergreen, cherry, citric acid, tartaric acid, menthol, lemon oil, citrus flavor, common salt, and other flavors known in the art.

The flavoring is advantageously chosen to give a combination of fast onset and long-lasting sweet taste and get a “round feeling” in the mouth with different textures or additives. Cooling agents can also be added in order to improve the mouth feeling and provide a synergy with flavors and sweetness. Various other materials may be present as coatings or to otherwise modify the physical form of the dosage unit. For instance, tablets or capsules may be coated with shellac, sugar or both.

In certain embodiments of the pharmaceutical compositions described herein include BHA, microcrystalline cellulose, croscarmellose, lactose monohydrate, hydroxypropyl cellulose and magnesium stearate. For example, the pharmaceutical compositions described herein can include 0.02% by weight of BHA, 20% by weight of microcrystalline cellulose, 3% by weight of croscarmellose, about 73-74% by weight of lactose monohydrate, 2% by weight of hydroxypropyl cellulose and 1% by weigh of magnesium stearate.

### Methods of Administration

The pharmaceutical compositions described herein can be formulated into pharmaceutical dosage forms that can be administered orally, intravenously, or parenterally. In certain embodiments the pharmaceutical compositions described herein are administered orally. In certain embodiments the pharmaceutical compositions described herein are formulated into capsules, tablets, pills, granules, puvules and the like. While the embodiments described herein contemplate any dosage form suitable for oral administration tablets and capsules including single or double shell gelatin capsules are especially preferred. Gelatin capsules can be hard or soft gelatin capsule. Other dosage forms may include suppositories, suspensions, liquids, creams, transdermal patches, and depots.

In certain embodiments, the pharmaceutical formulations described herein are formed into oral dosage forms that include a therapeutically effective amount of a CB1 inverse agonist, such as 3-[(1S)-1-(1-{(S)-(4-chlorophenyl)[3-(5-oxo-4,5-dihydro-1,3,4-oxadiazol-2-yl)phenyl]methyl}azetidin-3-yl)-2-fluoro-2-methylpropyl]-5-fluorobenzonitrile, in an amount of about 0.001 mg or less to about 200 mg or more, or preferably from about 0.01 mg to about 100 mg or preferably from about 0. 1 mg to about 50 mg. Preferably, the dosage range will be between about 0.5 mg to about 6 mg of the CB1 inverse agonists such as 3-[(1S)-1-(1-{(S)-(4-chlorophenyl)[3-(5-oxo-4,5-dihydro-1,3,4-oxadiazol-2-yl)phenyl]methyl}azetidin-3-yl)-2-fluoro-2-methylpropyl]-5-fluorobenzonitrile per patient per day.

By way of example, a particularly preferred solid oral dosage form may contain 3-[(1S)-1-(1-{(S)-(4-chlorophenyl)[3-(5-oxo-4,5-dihydro-1,3,4-oxadiazol-2-yl)phenyl]methyl}azetidin-3-yl)-2-fluoro-2-methylpropyl]-5-fluorobenzonitrile in a dosage amount of about 0.1 mg, 0.25 mg, 0.5 mg, about 1 mg, about 1.5 mg, about 2 mg, about 2.5 mg, 3 mg, about 3.5 mg, about 4 mg, about 4.5 mg, about 5 mg, about 5.5 mg, or about 6 mg. Of particular interest are 0.5 mg to 6 mg 3-[(1S)-1-(1-{(S)-(4-chlorophenyl)[3-(5-oxo-4,5-dihydro-1,3,4-oxadiazol-2-yl)phenyl]methyl}azetidin-3-yl)-2-fluoro-2-methylpropyl]-5-fluorobenzonitrile capsules or tablets. Of course, it should be appreciated that a particular unit dosage form and amount can be selected to accommodate the desired frequency of administration used to achieve a specified daily dosage and therapeutic effect. For example, oral dosage forms described herein may be administered to individuals on a regimen of one, two or more doses per day, at any time of the day.

It should be noted that the dosage amount may vary from patient to patient depending upon the nature and severity of the condition, the patient's weight, special diets being followed by a patient, concurrent medication, and other factors, recognized by those skilled in the art. Based upon the foregoing, precise dosages depend on the condition of the patient and are determined by discretion of a skilled clinician.

### Methods of Making

In certain embodiments described herein the pharmaceutical compositions, such as the capsules, are made by dissolving 3-[(1S)-1-(1-{(S)-(4-chlorophenyl)[3-(5-oxo-4,5-dihydro-1,3,4-oxadiazol-2-yl)phenyl]methyl}azetidin-3-yl)-2-fluoro-2-methylpropyl]-5-fluorobenzonitrile in a triglyceride. Depending on the triglyceride used, the triglyceride may be heated or stirred prior to or during the addition of 3-[(1S)-1-(1-{(S)-(4-chlorophenyl)[3-(5-oxo-4,5-dihydro-1,3,4-oxadiazol-2-yl)phenyl]methyl}azetidin-3-yl)-2-fluoro-2-methylpropyl]-5-fluorobenzonitrile.

In certain embodiments described herein an antioxidant can be added to the triglyceride before or after the addition of 3-[(S)-1-(1-{(S)-(4-chlorophenyl)[3-(5-oxo-4,5-dihydro-1,3,4-oxadiazol-2-yl)phenyl]methyl}azetidin-3-yl)-2-fluoro-2-methylpropyl]-5-fluorobenzonitrile.

As an example the following method was used to form oral dosage form of the pharmaceutical compositions of the invention:

1) IMWITOR was melted and added to an appropriate size vessel and stirred using an overhead mixer with an impeller;

2) BHA was added to the IMWITOR and the solution was mixed to obtain a homogeneous mixture;

3) 3-[(1S)-1-(1-{(S)-(4-chlorophenyl)[3-(5-oxo-4,5-dihydro-1,3,4-oxadiazol-2-yl)phenyl]methyl}azetidin-3-yl)-2-fluoro-2-methylpropyl]-5-fluorobenzonitrile was added slowly and the mixture was further mixed until the 3-[(1S)-1-(1-{(S)-(4-chlorophenyl)[3-(5-oxo-4,5-dihydro-1,3,4-oxadiazol-2-yl)phenyl]methyl}azetidin-3-yl)-2-fluoro-2-methylpropyl]-5-fluorobenzonitrile was completely dissolved;

4) the finished solution was dispensed into Size 0 capsules and banded.

In still other embodiments, described herein the pharmaceutical compositions, such as the tablets, are made by a wet granulation process. For example, granulation mixture can contain fillers, binders, disintegrating agents, antioxidants and diluents. The resulting granules can then be lubricated by adding a lubricant to the granules and compressed into tablets.

In certain embodiments the granulation mixture contains microcrystalline cellulose, lactose, hydroxypropyl cellulose, croscarmellose sodium and butyl hydroxyanisole (BHA). The granulation solvent is a mixture of ethanol and water. The resulting granules are lubricated with magnesium stearate and compressed.

### Methods of Treatment

In general, the pharmaceutical compositions described herein, stabilize and improve the bioavailability of CB1 inverse agonists, specifically, 3-[(1S)-1-(1-{(S)-(4-chlorophenyl)[3-(5-oxo-4,5-dihydro-1,3,4-oxadiazol-2-yl)phenyl]methyl}azetidin-3-yl)-2-fluoro-2-methylpropyl]-5-fluorobenzonitrile. CB1 inverse agonists are useful as centrally acting drugs in the treatment of psychosis, memory deficits, cognitive disorders, Alzheimer's disease, migraine, neuropathy, neuro-inflammatory disorders including multiple sclerosis and Guillain-Barre syndrome and the inflammatory sequelae of viral encephalitis, cerebral vascular accidents, and head trauma, anxiety disorders, stress, epilepsy, Parkinson's disease, movement disorders, schizophrenia, substance abuse disorders, particularly to opiates, alcohol, marijuana, and nicotine, smoking cessation and for the treatment of obesity or eating disorders associated with excessive food intake and complications associated therewith, including left ventricular hypertrophy.

CB1 inverse agonists are also useful for the treatment of constipation and chronic intestinal pseudo-obstruction, as well as treating cirrhosis of the liver, non-alcoholic fatty liver disease (NAFLD), non-alcoholic steatohepatitis (NASH), and asthma.

Thus the pharmaceutical compositions described herein are useful for treatment or, in some cases, prevention psychosis, memory deficits, cognitive disorders, Alzheimer's disease, migraine, neuropathy, neuro-inflammatory disorders including multiple sclerosis and Guillain-Barre syndrome and the inflammatory sequelae of viral encephalitis, cerebral vascular accidents, and head trauma, anxiety disorders, stress, epilepsy, Parkinson's disease, movement disorders, schizophrenia, substance abuse disorders, particularly to opiates, alcohol, marijuana, and nicotine, smoking cessation and for the treatment of obesity or eating disorders associated with excessive food intake and complications associated therewith, including left ventricular hypertrophy.

The pharmaceutical composition described herein are also useful for the treatment of constipation and chronic intestinal pseudo-obstruction, as well as treating cirrhosis of the liver, non-alcoholic fatty liver disease (NAFLD), non-alcoholic steatohepatitis (NASH), and asthma.

The pharmaceutical compositions described herein can be used to treat humans and other mammalian species, including canines and felines.

For example, in certain embodiments the pharmaceutical compositions described herein are useful for treating or preventing obesity by administering to a subject in need thereof a composition comprising 3-[(1S)-1-(1-{(S)-(4-chlorophenyl)[3-(5-oxo-4,5-dihydro-1,3,4-oxadiazol-2-yl)phenyl]methyl}azetidin-3-yl)-2-fluoro-2-methylpropyl]-5-fluorobenzonitrile.

Methods of treating or preventing obesity and conditions associated with obesity refer to the administration of the pharmaceutical formulations described herein to reduce or maintain the body weight of an obese subject or to reduce or maintain the body weight of an individual at risk of becoming obese. One outcome of treatment may be reducing the body weight of an obese subject relative to that subject's body weight immediately before the administration of the compounds or combinations of the present invention. Another outcome of treatment may be preventing body weight, regain of body weight previously lost as a result of diet, exercise, or pharmacotherapy and preventing weight gain from cessation of smoking. Another outcome of treatment may be decreasing the occurrence of and/or the severity of obesity-related diseases. Yet another outcome of treatment may be decreasing the risk of developing diabetes in an overweight or obese subject. The treatment may suitably result in a reduction in food or calorie intake by the subject, including a reduction in total food intake, or a reduction of intake of specific components of the diet such as carbohydrates or fats; and/or the inhibition of nutrient absorption; and/or the inhibition of the reduction of metabolic rate; and in weight reduction in patients in need thereof. The treatment may also result in an alteration of metabolic rate, such as an increase in metabolic rate, rather than or in addition to an inhibition of the reduction of metabolic rate; and/or in minimization of the metabolic resistance that normally results from weight loss.

Prevention of obesity and obesity-related disorders refers to the administration of the pharmaceutical formulations described herein to reduce or maintain the body weight of a subject at risk of obesity. One outcome of prevention may be reducing the body weight of a subject at risk of obesity relative to that subject's body weight immediately before the administration of the compounds or combinations of the present invention. Another outcome of prevention may be preventing body weight regain of body weight previously lost as a result of diet, exercise, or pharmacotherapy. Another outcome of prevention may be preventing obesity from occurring if the treatment is administered prior to the onset of obesity in a subject at risk of obesity. Another outcome of prevention may be decreasing the occurrence and/or severity of obesity-related disorders if the treatment is administered prior to the onset of obesity in a subject at risk of obesity. Moreover, if treatment is commenced in already obese subjects, such treatment may prevent the occurrence, progression or severity of obesity-related disorders, such as, but not limited to, arteriosclerosis, Type 2 diabetes, polycystic ovary disease, cardiovascular diseases, osteoarthritis, dermatological disorders, hypertension, insulin resistance, hypercholesterolemia, hypertriglyceridemia, and cholelithiasis.

In other embodiments the pharmaceutical compositions described herein are useful for treating or preventing diabetes by administering to a subject in need thereof a composition comprising 3-[(1S)-1-(1-{(S)-(4-chlorophenyl)[3-(5-oxo-4,5-dihydro-1,3,4-oxadiazol-2-yl)phenyl]methyl}azetidin-3-yl)-2-fluoro-2-methylpropyl]-5-fluorobenzonitrile.

In other embodiments the pharmaceutical compositions described herein are useful for treating or preventing Alzheimer's Disease and related disorders by administering to a subject in need thereof a composition comprising 3-[(S)-1-(1-{(S)-(4-chlorophenyl)[3-(5-oxo-4,5-dihydro-1,3,4-oxadiazol-2-yl)phenyl]methyl}azetidin-3-yl)-2-fluoro-2-methylpropyl]-5-oxo-fluorobenzonitrile and a triglyceride, such as fractionated coconut oils such as known and commercially available under the trade name MIGLYOL, including the products: MIGLYOL 810; MIGLYOL 812; MIGLYOL 818; and CAPTEX 355.

Treatment of Alzheimer's Disease and Alzheimer's Disease-related disorders refers to the administration of the compositions of the present invention to treat Alzheimer's Disease and to treat early, intermediate and late stage dementia of Alzheimer's type. One outcome of treatment may be treating Alzheimer's Disease. Another outcome of treatment may be controlling Alzheimer's Disease. Another outcome of treatment may be ameliorating Alzheimer's disease. Another outcome of treatment may be reducing the risk of Alzheimer's Disease. Another outcome of treatment may be the prevention of the dementia of Alzheimer's type. Another outcome of treatment may be the prevention of the early stage dementia of Alzheimer's type. Another outcome of treatment may be the prevention of intermediate stage dementia of Alzheimer's type. Another outcome of treatment may be the prevention of late stage dementia of Alzheimer's type. Another outcome of treatment may be treating an Alzheimer's Disease related disorder. Another outcome of treatment may be prevention or delay of the onset of Alzheimer's disease. Another outcome of treatment may be halting or slowing of the patient's cognitive decline. Another outcome of treatment may be halting or slowing of the progress of cognitive impairment. Another outcome of treatment may be enhanced clearance of Aβ from the brain. Another outcome of treatment may be preventing or delaying the onset of dementia associated with Alzheimer's disease. Another outcome of treatment may be treating age-related cognitive decline or mild cognitive impairment. Another outcome of treatment may be preventing, retarding or arresting any further age-related cognitive decline or progression of mild cognitive impairment. Another outcome of treatment may be preventing or delaying the onset of Alzheimer's disease. Another outcome of treatment may be preventing or delaying the onset of dementia associated with Alzheimer's disease. Another outcome of treatment may be preventing, retarding or arresting the accumulation of insoluble Aβ in the brain. Moreover, if treatment is commenced in early stage dementia of Alzheimer's type in Alzheimer's disease subjects, such treatment may prevent the occurrence, progression or severity of Alzheimer's disease and/or Alzheimer's disease related disorders. Treatment includes prevention of Alzheimer's disease and Alzheimer's disease related disorders.

Prevention of Alzheimer's Disease and Alzheimer's Disease-related disorders refers to the administration of the compositions of the present invention to prevent Alzheimer's Disease and to prevent early, intermediate and late stage dementia of Alzheimer's type. One outcome of prevention may be reducing the risk of Alzheimer's Disease. Another outcome of prevention may be the prevention of the dementia of Alzheimer's type. Another outcome of prevention may be the prevention of the early stage dementia of Alzheimer's type. Another outcome of prevention may be the prevention of intermediate stage dementia of Alzheimer's type. Another outcome of prevention may be the prevention of late stage dementia of Alzheimer's type. Another outcome of prevention may be prevention of Alzheimer's Disease related disorders. Another outcome of prevention may be prevention or delay of the onset of Alzheimer's disease. Another outcome of prevention may be halting or slowing of the patient's cognitive decline. Another outcome of prevention may be halting or slowing of the progress of cognitive impairment. Another outcome of prevention may be preventing or delaying the onset of dementia associated with Alzheimer's disease. Another outcome of prevention may be preventing, retarding or arresting any further age-related cognitive decline or progression of mild cognitive impairment. Another outcome of prevention may be preventing or delaying the onset of Alzheimer's disease. Another outcome of prevention may be preventing the onset of dementia associated with Alzheimer's disease. Another outcome of prevention may be preventing or retarding the accumulation of insoluble Aβ in the brain.

Alzheimer's Disease is described in the Diagnostic and Statistical Manual of Mental Disorders, 4th ed., published by the American Psychiatric Association (DSM-IV) (e.g. pages 139-143). Diagnostic criteria for Alzheimer's disease includes the development of multiple cognitive deficits in a patient manifested by both (1) memory impairment (impaired ability to learn new information or to recall previously learned information), and (2) one (or more) of the following cognitive disturbances (a) aphasia (language disturbance), (b) apraxia (impaired ability to carry out motor activities despite intact motor function), (c) agnosia (failure to recognize or identify objects despite intact sensory function) and (d) disturbances in executing functioning (i.e. planning, organizing, sequencing, abstracting). Such cognitive deficits are not characterized as being due to any of the following: (1) other central nervous system conditions that cause progressive deficits in memory and cognition (e.g. cerebrovascular disease, Parkinson's disease, Huntington's disease, subdural hematoma, normal-pressure hydrocephalus, brain tumor), (2) systemic conditions that are known to cause dementia (e.g. hypothyroidism, vitamin B12 or folic acid deficiency, niacin deficiency, hypercalcemia, neurosyphilis, HIV infection), (3) substance-induced conditions.

Alzheimer's Disease-related disorders are associated with, caused by, or result from Alzheimer's Disease or precede Alzheimer's Disease. Examples of Alzheimer's Disease-related disorders include, but are not limited to, dementia, age related cognitive decline, mild cognitive impairment, memory impairment, aphasia, apraxia, agnosia and disturbances in executing functioning, memory deficits, memory impairment, and cognitive disorders. In one embodiment of the present invention, Alzheimer's Disease related disorders are selected from: dementia, age related cognitive decline, mild cognitive impairment, memory impairment, aphasia, apraxia, agnosia and disturbances in executing functioning. In another embodiment of the present invention, Alzheimer's Disease related disorders are selected from: dementia, age related cognitive decline, and mild cognitive impairment. In another embodiment of the present invention, the Alzheimer's Disease related disorder is dementia.

Age-related cognitive decline and mild cognitive impairment (MCI) are conditions in which a memory deficit is present, but other diagnostic criteria for dementia are absent (Santacruz and Swagerty, American Family Physician, 63 (2001), 703-13). (See also “The ICD-10 Classification of Mental and Behavioural Disorders”, Geneva: World Health Organisation, 1992, 64-5). When used herein, age-related cognitive decline is characterized by a decline of at least six months' duration in at least one of: memory and learning; attention and concentration; thinking; language; and visuospatial functioning and a score of more than one standard deviation below the norm on standardized neuropsychologic testing such as the MMSE. In particular, there may be a progressive decline in memory. In the more severe condition mild cognitive impairment (MCI), the degree of memory impairment is outside the range considered normal for the age of the patient but Alzheimer's disease (AD) is not present. The differential diagnosis of MCI and mild AD is described by Petersen et al., Arch. Neurol., 56 (1999), 303-8. In the same article, Petersen et al. disclose that the patients suffering from MCI typically experience a progressive increase in cognitive impairment and in many cases develop AD. Further information on the differential diagnosis of MCI is provided by Knopman et al, Mayo Clinic Proceedings, 78 (2003), 1290-1308. In a study of elderly subjects, Tuokko et al (Arch, Neurol., 60 (2003) 577-82) found that those exhibiting MCI at the outset had a three-fold increased risk of developing dementia within 5 years.

Grundman et al (J. Mol. Neurosci., 19 (2002), 23-28) report that lower baseline hippocampal volume in MCI patients is a prognostic indicator for subsequent AD. Similarly, Andreasen et al (Acta Neurol. Scand, 107 (2003) 47-51) report that high CSF levels of total tau, high CSF levels of phospho-tau and lowered CSF levels of Aβ42 are all associated with increased risk of progression from MCI to AD.

Age-related cognitive decline and mild cognitive impairment are distinct from the significant cognitive deficit that sometimes results from cerebral or systemic diseases and traumas, such as stroke, concussion, or major dysfunction of the pituitary.

### Combination Therapy

The pharmaceutical compositions described herein can be used in combination with additional therapeutic agents. In certain embodiments, the pharmaceutical formulations described herein and the additional therapeutic agent may be in separate dosage forms and may be administered via the same or a different route. For example, the pharmaceutical formulations described herein may be administered orally while the additional therapeutic agent is administered parenterally or, alternatively, both may be administered orally. Additionally, the pharmaceutical formulations described herein and the additional therapeutic agent may be administered contemporaneously or sequentially.

In other embodiments, the pharmaceutical formulations described herein can include the additional therapeutic agent. Examples of additional therapeutic agents that may be included with the pharmaceutical formulations described herein include, but are not limited to: antipsychotic agents, cognition enhancing agents, anti-migraine agents, anti-asthmatic agents, anti-inflammatory agents, axiolytics, anti-Parkinson's agents, anti-epileptics, anorectic agents and

(a) insulin sensitizers including (i) PPARγ antagonists such as glitazones (e.g. ciglitazone; darglitazone; englitazone; isaglitazone (MCC-555); pioglitazone; rosiglitazone; troglitazone; tularik; BRL49653; CLX-0921; 5-BTZD), GW-0207, LG-100641, and LY-300512, and the like), and compounds disclosed in WO97/10813, 97/27857, 97/28115, 97/28137, and 97/27847; (ii) biguanides such as metformin and phenformin;

(b) insulin or insulin mimetics, such as biota, LP-100, novarapid, insulin detemir, insulin lispro, insulin glargine, insulin zinc suspension (lente and ultralente); Lys-Pro insulin, GLP-1 (73-7) (insulintropin); and GLP-1 (7-36)-NH2);

(c) sulfonylureas, such as acetohexamide; chlorpropamide; diabinese; glibenclamide; glipizide; glyburide; glimepiride; gliclazide; glipentide; gliquidone; glisolamide; tolazamide; and tolbutamide;

(d) α-glucosidase inhibitors, such as acarbose, adiposine; camiglibose; emiglitate; miglitol; voglibose; pradimicin-Q; salbostatin; CKD-711; MDL-25,637; MDL-73,945; and MOR 14, and the like;

(e) cholesterol lowering agents such as (i) HMG-CoA reductase inhibitors (atorvastatin, itavastatin, fluvastatin, lovastatin, pravastatin, rivastatin, rosuvastatin, simvastatin, and other statins), (ii) bile acid absorbers/sequestrants, such as cholestyramine, colestipol, dialkylaminoalkyl derivatives of a cross-linked dextran; COLESTID®; LOCHOLEST®, and the like, (ii) nicotinyl alcohol, nicotinic acid or a salt thereof, (iii) proliferator-activater receptor α agonists such as fenofibric acid derivatives (gemfibrozil, clofibrate, fenofibrate and benzafibrate), (iv) inhibitors of cholesterol absorption such as stanol esters, beta-sitosterol, sterol glycosides such as tiqueside; and azetidinones such as ezetimibe, and the like; and acyl CoA: cholesterol acyltransferase (ACAT) inhibitors such as avasimibe, and melinamide, (v) anti-oxidants, such as probucol, (vi) vitamin E, and (vii) thyromimetics;

(f) PPARα agonists such as beclofibrate, benzafibrate, ciprofibrate, clofibrate, etofibrate, fenofibrate, and gemfibrozil; and other fibric acid derivatives, such as ATROMID®, LOPID® and TRICOR®, and the like, and PPARα agonists as described in WO 97/36579 by Glaxo;

(g) PPAR6 agonists, such as those disclosed in WO97/28149; and

(h) anti-obesity agents, such as (1) growth hormone secretagogues, growth hormone secretagogue receptor agonists/antagonists, such as NN703, hexarelin, MK-0677, SM-130686, CP-424,391, L-692,429 and L-163,255, and such as those disclosed in U.S. Pat. Nos. 5,536,716, and 6,358,951, U.S. Patent Application Nos. 2002/049196 and 2002/022637, and PCT Application Nos. WO 01/56592 and WO 02/32888; (2) protein tyrosine phosphatase-1B (PTP-1B) inhibitors; (3) cannabinoid receptor ligands, such as cannabinoid CB1 receptor antagonists or inverse agonists, such as rimonabant (Sanofi Synthelabo), and SR-147778 (Sanofi Synthelabo), and those disclosed in U.S. Pat. Nos. 5,532,237, 4,973,587, 5,013,837, 5,081,122, 5,112,820, 5,292,736, 5,624,941, 6,028,084, PCT Application Nos. WO 96/33159, WO 98/33765, WO98/43636, WO98/43635, WO 01/09120, WO98/31227, WO98/41519, WO98/37061, WO00/10967, WO00/10968, WO97/29079, WO99/02499, WO 01/58869, and EPO Application No. EP-658546; (4) anti-obesity serotonergic agents, such as fenfluramine, dexfenfluramine, phentermine, and sibutramine; (5) P3-adrenoreceptor agonists, such as AD9677/TAK677 (Dainippon/Takeda), CL-316,243, SB 418790, BRL-37344, L-796568, BMS-196085, BRL-3 513 5A, CGP 12177A, BTA-243, Trecadrine, Zeneca D7114, SR 59119A, and such as those disclosed in U.S. Patent Application Nos. 5,705,515, and U.S. Pat. No. 5,451,677 and PCT Patent Publications WO94/18161, WO95/29159, WO97/46556, WO98/04526 and WO98/32753, WO01/74782, and WO 02/32897; (6) pancreatic lipase inhibitors, such as orlistat (Xenical®), Triton WR1339, RHC80267, lipstatin, tetrahydrolipstatin, teasaponin, diethylumbelliferyl phosphate, and those disclosed in PCT Application No. WO 01/77094; (7) neuropeptide Y1 antagonists, such as BIBP3226, J-115814, BIBO 3304, LY-357897, CP-671906, GI-264879A, and those disclosed in U.S. Pat. No. 6,001,836, and PCT Patent Publication Nos. WO 96/14307, WO 01/23387, WO 99/51600, WO 01/85690, WO 01/85098, WO 01/85173, and WO 01/89528; (8) neuropeptide Y5 antagonists, such as GW-569180A, GW-594884A, GW-587081X, GW-548118X, FR226928, FR 240662, FR252384, 1229U91, GI-264879A, CGP71683A, LY-377897, PD-160170, SR-120562A, SR-120819A and JCF-104, and those disclosed in U.S. Pat. Nos. 6,140,354, 6,191,160, 6,313,298, 6,337,332, 6,329,395, 6,326,375, 6,335,345, and 6,340,683, European Patent Nos. EP-01010691, and EP-01044970, and PCT Patent Publication Nos. WO 97/19682, WO 97/20820, WO 97/20821, WO 97/20822, WO 97/20823, WO 98/27063, WO 00/64880, WO 00/68197, WO 00/69849, WO 01/09120, WO 01/14376, WO 01/85714, WO 01/85730, WO 01/07409, WO 01/02379, WO 01/02379, WO 01/23388, WO 01/23389, WO 01/44201, WO 01/62737, WO 01/62738, WO 01/09120, WO 02/22592, WO 0248152, and WO 02/49648; (9) melanin-concentrating hormone (MCH) receptor antagonists, such as those disclosed in WO 01/21577 and WO 01/21169; (10) melanin-concentrating hormone 1 receptor (MCH1R) antagonists, such as T-226296 (Takeda), and those disclosed in PCT Patent Application Nos. WO 01/82925, WO 01/87834, WO 02/06245, WO 02/04433, and WO 02/51809, and Japanese Patent Application No. JP 13226269; (11) melanin-concentrating hormone 2 receptor (MCH2R) agonist/antagonists; (12) orexin-1 receptor antagonists, such as SB-334867-A, and those disclosed in PCT Patent Application Nos. WO 01/96302, WO 01/68609, WO 02/51232, and WO 02/51838; (13) serotonin reuptake inhibitors such as fluoxetine, paroxetine, and sertraline, and those disclosed in U.S. Patent Application No. 6,365,633, and PCT Patent Application Nos. WO 01/27060 and WO 01/162341; (14) melanocortin agonists, such as Melanotan II or those described in WO 99/64002 and WO 00/74679; (15) other Mc4r (melanocortin 4 receptor) agonists, such as CHIR86036 (Chiron), ME-10142, and ME-10145 (Melacure), and those disclosed in PCT Application Nos. WO 01/991752, WO 01/74844, WO 02/12166, WO 02/11715, and WO 02/12178; (16) 5HT-2 agonists; (17) 5HT2C (serotonin receptor 2C) agonists, such as BVT933, DPCA37215, WAY161503, R-1 065, and those disclosed in U.S. Pat. No. 3,914,250, and PCT Application Nos. WO 02/36596, WO 02/48124, WO 02/10169, WO 01/66548, WO 02/44152, WO 02/51844, WO 02/40456, and WO 02/40457; (18) galanin antagonists; (19) CCK agonists; (20) CCK-A (cholecystokinin -A) agonists, such as AR-R 15849, GI 181771, JMV-180, A-71378, A-71623 and SR146131, and those discribed in U.S. Pat. No. 5,739,106; (21) GLP-1 agonists; (22) corticotropin-releasing hormone agonists; (23) histamine receptor-3 (H3) modulators; (24) histamine receptor-3 (H3) antagonists/inverse agonists, such as hioperamide, 3-(1H-imidazol-4-yl)propyl N-(4-pentenyl)carbamate, clobenpropit, iodophenpropit, imoproxifan, GT2394 (Gliatech), and those described and disclosed in PCT Application No. WO 02/15905, and O-[3-(1H-imidazol-4-yl)propanol]-carbamates (Kiec-Kononowicz, K. et al., Pharmazie, 55:349-55 (2000)), piperidine-containing histamine H3-receptor antagonists (Lazewska, D. et al., Pharmazie, 56:927-32 (2001), benzophenone derivatives and related compounds (Sasse, A. et al., Arch. Pharm.(Weinheim) 334:45-52 (2001)), substituted N-phenylcarbamates (Reidemeister, S. et al., Pharmazie, 55:83-6 (2000)), and proxifan derivatives (Sasse, A. et al., J. Med. Chem. 43:3335-43 (2000)); (25) α-hydroxy steroid dehydrogenase-1 inhibitors (α-HSD-1); 26) PDE (phosphodiesterase) inhibitors, such as theophylline, pentoxifylline, zaprinast, sildenafil, amrinone, milrinone, cilostamide, rolipram, and cilomilast; (27) phosphodiesterase-3B (PDE3B) inhibitors; (28) NE (norepinephrine) transport inhibitors, such as GW 320659, despiramine, talsupram, and nomifensine; (29) ghrelin antagonists, such as those disclosed in PCT Application Nos. WO 01/87335, and WO 02/08250; (30) leptin, including recombinant human leptin (PEG-OB, Hoffman La Roche) and recombinant methionyl human leptin (Amgen); (31) leptin derivatives, such as those disclosed in U.S. Pat. Nos. 5,552,524, 5,552,523, 5,552,522, 5,521,283, and PCT International Publication Nos. WO 96/23513, WO 96/23514, WO 96/23515, WO 96/23516, WO 96/23517, WO 96/23518, WO 96/23519, and WO 96/23520; (32) BRS3 (bombesin receptor subtype 3) agonists; (33) CNTF (Ciliary neurotrophic factors), such as GI-181771 (Glaxo-SmithKline), SR146131 (Sanofi Synthelabo), butabindide, PD170,292, and PD 149164 (Pfizer); (34) CNTF derivatives, such as axokine (Regeneron), and those disclosed in PCT Application Nos. WO 94/09134, WO 98/22128, and WO 99/43813; (35) monoamine reuptake inhibitors, such as those disclosed in PCT Application Nos. WO 01/27068, and WO 01/62341; (36) UCP-1 (uncoupling protein-1), 2, or 3 activators, such as phytanic acid, 4-[(E)-2-(5,6,7, 8-tetrahydro-5,5,8,8-tetramethyl-2-napthalenyl)-1-propenyl]benzoic acid (TTNPB), retinoic acid, and those disclosed in PCT Patent Application No. WO 99/00123; (37) thyroid hormone P agonists, such as KB-2611 (KaroBioBMS), and those disclosed in PCT Application No. WO 02/15845, and Japanese Patent Application No. JP 2000256190; (38) FAS (fatty acid synthase) inhibitors, such as Cerulenin and C75; (39) DGAT1 (diacylglycerol acyltransferase 1) inhibitors; (40) DGAT2 (diacylglycerol acyltransferase 2) inhibitors; (41) ACC2 (acetyl-CoA carboxylase-2) inhibitors; (42) glucocorticoid antagonists; (43) acyl-estrogens, such as oleoyl-estrone, disclosed in del Mar-Grasa, M. et al., Obesity Research, 9:202-9 (2001); (44) dipeptidyl peptidase IV (DP-IV) inhibitors, such as stiagliptin, algoliptin, saxagliptin, denagliptin, melogliptin, vildagliptin, BI-1356 and PF-7342000, DPP-728, P 3298, TSL 225, valine pyrrolidide, TMC-2A/2B/2C, CD-26 inhibitors, FE 999011, P9310/K364, VIP 0177, NVPLAF237, DPP4, SDZ 274-444; and the compounds disclosed in EP 1258476, EP 1258480, and WO 03/000180; (45) fatty acid transporter inhibitors; (46) dicarboxylate transporter inhibitors; (47) glucose transporter inhibitors; (48) phosphate transporter inhibitors; (49) Metformin (Glucophage®); and (50) Topiramate (Topimax®);

(i) Alzheimer medications such as memantine, galantamine, rivastigmine, donepexil and tacrine.

Examples of other anti-obesity agents that can be employed in combination with a compound of Formula I are disclosed in “Patent focus on new anti-obesity agents,” Exp. Opin. Ther. Patents, 10: 819-831 (2000); “Novel anti-obesity drugs,” Exp. Opin. Invest. Drugs, 9: 1317-1326 (2000); and “Recent advances in feeding suppressing agents: potential therapeutic strategy for the treatment of obesity, Exp. Opin. Ther. Patents, 11: 1677-1692 (2001). The role of neuropeptide Y in obesity is discussed in Exp. Opin. Invest. Drugs, 9: 1327-1346 (2000). Cannabinoid receptor ligands are discussed in Exp. Opin. Invest. Drugs, 9: 1553-1571 (2000).

For example, in certain embodiments the pharmaceutical formulations described herein include a solid dispersion of 3-[(1S)-1-(1-{(S)-(4-chlorophenyl)[3-(5-oxo-4,5-dihydro-1,3,4-oxadiazol-2-yl)phenyl]methyl}azetidin-3-yl)-2-fluoro-2-methylpropyl]-5-fluorobenzonitrile, a polymer and optionally a surfactant, and a DPP-4 inhibitor such as sitagliptin or a pharmaceutically acceptable salt thereof such as, sitagliptin phosphate.

### Examples

### Example 1

### Method of Making 3-[(1S)-1-(1-{(S)-(4-chlorophenyl)[3-(5-oxo-4,5-dihydro-1,3,4-oxadiazol-2-yl)phenyl]methyl}azetidin-3-yl)-2-fluoro-2-methylpropyl]-5-fluorobenzonitrile

### Step 1 N-[(1E)-(3-cyanophenyl)methylene]-2-methylpropane-2-(R)sulfinamide

A solution of 19.0 g (157 mmole) of (R)-(+)-2-methylpropane-2-sulfinamide and 89.0 g (314 mmole) of titanium tetraisopropoxide in CH₂Cl₂ was stirred at room temperature for 10 min. Then a solution of 21.6 g (165 mmole) of 3-formylbenzonitrile in 10 mL CH₂Cl₂ was added, and the solution was stirred at room temperature. After 18 h, the reaction was quenched by the addition of 30 mL brine and the solution was rapidly stirred for 15 min. The mixture was filtered through a pad of CELITE and the residue was washed with 300 mL of CH₂Cl₂. The combined organic extracts were washed with brine, dried over Na₂SO₄ and concentrated. The residue was filtered through a pad of silica gel using 20% ethyl acetate-hexane to afford the title compound; ¹H-NMR(CDCl₃) δ 1.31 (s, 9H), 7.65 (t, 1H, J=7.8 Hz), 7.82 (d, 1H, J=7.8 Hz), 8.07 (d, 1H, J=7.8 Hz), 8.20 (s, 1H), 8.62 (s, 1H); Mass Spectrum: m/e=235 (M+1).

### Step 2 N-[(S)-(4-chlorophenyl)(3-cyanophenyl)methyl]-2-methylpropane-2-(R)-sulfinamide

A solution of 20 g (85.4 mmole) of N-[(1E)-(3-cyanophenyl)methylene]-2-methylpropane-2-(R)sulfonamide in 1000 mL toluene and 400 mL ether was cooled to −60° C. in a dry ice-acetone bath. Then 170 mL of a 1M solution of 4-chlorophenylmagnesium bromide in ether was added at a rate such that the temperature remained between −40° C. and −30° C. and the reaction was stirred at −30° C. for 6 h. The reaction was quenched by addition of 300 mL of saturated NH₄Cl solution and the layers were separated. The organic layer was washed with three 300 mL aliquots of saturated NH₄Cl solution and brine, then was dried over Na₂SO₄ and concentrated. The residue was filtered through a pad of silica gel using 10 to 30% ethyl acetate hexane to afford the title compound with de >99% as determined by analytical ChiralPak AD column; ¹H-NMR(CDCl₃) δ 1.27 (s, 9H), 3.76 (s, 1H), 5.65 (d, 1H, J=2.3 Hz), 7.24-7.7 (m, 8H).

### Step 3 3-[(S)-amino(4-chlorophenyl)methyl]benzonitrile hydrochloride

To a solution of 850 mg (2.45 mmole) of N-[(S)-(4-chlorophenyl)(3-cyanophenyl)methyl]-2-methylpropane-2-(R)-sulfinamide in 20 mL of CH₃OH was added 2.5 mL of 4M HCL in dioxane. The solution was stirred at room temperature for 45 min, then was diluted with 40 mL ether. The solids were collected by filtration to afford the title compound as a white solid; ¹H-NMR(CDCl₃) δ 1.6 (s, 2H, br), 5.24 (s, 1H), 7.24-7.78 (m, 8H).

### Step 4 3-[(S)-(4-chlorophenyl)(3-hydroxyazetidin-1-yl)methyl]benzonitrile

To a mixture of 20.27 g (72.6 mmole) of 3-[(S)-[(3-chloro-2-hydroxypropyl)amino](4-chlorophenyl)methyl]benzonitrile hydrochloride and 21.3 g (245 mmole) of NaHCO₃ in 600 mL of isopropanol was added 14.4 mL (174 mmole) of epibromohydrin. The mixture was heated to reflux for 24 h, then was cooled and concentrated. The residue was partitioned between 750 mL portions of ether and water and the aqueous layer was washed with two 500 mL portions of ether. The combined organic extracts were washed with brine, dried over MgSO₄ and concentrated. The residue was purified by flash chromatography using 10-20% ethyl acetate in hexane to afford the title compound as a clear oil; ¹H-NMR(CDCl₃) δ 1.6 (s, 2H, br), 5.24 (s, 1H), 7.24-7.78 (m, 8H).2.89 (m, 2H), 3.54 (m, 2H), 4.39 (s, 1H), 4.52 (m, 1H), 7.2-7.8 (m, 8H).

### Step 5 3-[(S)-(4-chlorophenyl)(3-oxoazetidin-1-yl)methyl]benzonitrile

To a solution of 21.1 mL (0.24 mol) of oxalyl chloride in 500 mL CH₂Cl₂, a solution of 34.2 mL (0.48 mol) of DMSO in 50 mL CH₂Cl₂ was added slowly at −78° C. After the reaction mixture was stirred for 30 min., a solution of 36.02 g (0.12 mol) of3-[(S)-(4-chlorophenyl)(3-hydroxyazetidin-1-yl)methyl]benzonitrile in 50 mL of CH₂Cl₂ was added and stirred for another 45 min. Then 82.8 mL (0.60 mol) of triethylamine was added and the mixture was stirred for 30 min. at −78° C. The mixture was warmed to room temperature and stirring continued for 30 min. Then the mixture was poured into 1000 mL of ether and 200 mL of aqueous NaHCO₃. The water layer was extracted with two 200 mL portions of ether. The combined organic layers were dried over Na₂SO₄ and concentrated. The residue was purified by silica gel chromatography with hexanes/ethyl acetate to afford the title compound as a white solid; ¹H-NMR(CDCl₃) δ 4.03-4.07(m, 4H), 4.65(s, 1H), 7.33-7.43 (m, 4H), 7.45 (t, 1H, J=7.8 Hz), 7.56 (d, 1H, J=7.5Hz), 7.72 (d, 1H, J=7.5Hz), 7.81 (s, 1H); Mass Spectrum: m/e=297, (M+1, ³⁵Cl), 299 (M+1, ³⁷Cl).

### Step 6 Methyl(3-bromo-5-fluorophenyl){1-[(S)-(4-chlorophenyl)(3-cyanophenyl)methyl]azetidin-3-ylidene}acetate

To a solution of 14.55 g (58.87 mmol) of methyl (3-bromo-5-fluorophenyl)acetate in 200 mL of THF at −78° C., was added a solution of 56.80 mL (56.80 mmol) (1M in THF) of LHMDS. After the reaction mixture was stirred for 30 min., a solution of 15.60 g (52.57 mmol) of 3-[(S)-(4-chlorophenyl)(3-oxoazetidin-1-yl)methyl]benzonitrile in 50 mL of THF was added and the mixture was stirred for 2.5 h at −78° C. Then 8.35 g (68.33 mmol) of DMAP, 14.65 mL (84.09 mmol) of DIEA, and 8.72 mL (110.38 mmol) of methanesulfonyl chloride were added, and the mixture was stirred for 1 h at −78° C. The mixture was then allowed to warm to room temperature and was stirred at room temperature for 12 h. The mixture was poured into 300 mL of ether and 100 mL of water. The water layer was extracted with ether (100 mL x2). The combined organic layer was dried over Na₂SO₄ and concentrated. The residue was purified by silica gel chromatography with hexanes/ethyl acetate to afford the title compound as a white solid; ¹H-NMR(CDCl₃) δ 3.75(s, 3H), 3.87(s, 2H), 4.23-4.29(m, 2H), 4.59(s, 1H), 6.92 (d, 1H, J=12 Hz), 7.18(s, 1H), 7.20 (d, 1H, J=12Hz), 7.31-7.38 (dd, 4H, J J₁ 1=28.4 Hz, J₂=8.5 Hz), 7.42 (t, 1H, J=7.7 Hz), 7.54 (d, 1H, J=7.6 Hz), 7.66 (d, 1H, J=7.6 Hz), 7.76 (s, 1H); Mass Spectrum: m/e=525, (M+1, ³⁵Cl ⁷⁹Br), 527 (M+1, ³⁷Cl⁷⁹Br/³⁵Cl⁸¹Br), 529 (M+1, ³⁷Cl ⁸¹Br).

### Step 7 methyl (2S)-(3-bromo-5-fluorophenyl){1-[(S)-(4-chlorophenyl)(3-cyanophenyl)methyl]azetidin-3-yl}acetate

To a solution of 27.15 g (51.64 mmol) of methyl (3-bromo-5-fluorophenyl){1-[(S)-(4-chlorophenyl) (3-cyanophenyl)methyl]azetidin-3-ylidene}acetate in 120 mL of THF and 220 mL of MeOH, were added small portions of sodium borohydride (total: 740 mg, 31.05 mmol) at −5-0° C. The mixture stirred at −5-0° C. and the reaction was followed by HPLC. Then the reaction was quenched with 2N HCl at 0° C. (to pH=7-7.5) and concentrated to remove the organic solvents. The residue was dissolved in 300 mL of CH₂Cl₂ and 300 mL water, and the layers were separated. The aqueous layer was extracted with CH₂Cl₂ (100 mL x2). The combined organic layer was dried over Na₂S0₄ and concentrated. The residue was separated by silica gel chromatography with cyclohexane/ethyl acetate to afford the methyl (2S)-(3-bromo-5-fluorophenyl){1-[(S)-(4-chlorophenyl)(3-cyanophenyl)methyl]azetidin-3-yl}acetate; ¹H-NMR(CDCl₃) δ 2.66(t, 1H, J=6.2 Hz), 2.92(dd, 1H, J₁=7.5 Hz, J₂=5.7 Hz), 3.08-3.16(m, 2H), 3.41(t, 1H, J=7.2 Hz), 3.69(s, 3H), 3.83(d, 1H, J=10.7 Hz), 4.34(s, 1H), 6.96 (d, 1H, J=8.9 Hz), 7.17(d, 1H, J=8.0 Hz), 7.20 (d, 1H, J=12 Hz), 7.27-7.32 (m, 5H), 7.39 (t, 1H, J=7.6 Hz), 7.50 (d, 1H, J=7.5 Hz), 7.60 (d, 1H, J=7.5 Hz), 7.70 (s, 1H); Mass Spectrum: m/e=527(M+1, ³⁵Cl ⁷⁹Br), 529 (M+1, ³⁷Cl⁷⁹Br/³⁵Cl ⁸¹Br), 531 (M+1, ³⁷Cl ⁸¹Br). Further elution of the column afforded methyl (2R)-(3-bromo-5-fluorophenyl) {1-[(S)-(4-chlorophenyl)(3-cyanophenyl)methyl]azetidin-3-yl}acetate; ¹H-NMR(CDCl₃) δ 2.67(t, 1H, J=6.2 Hz), 2.90(dd, 1H, J₁=7.3 Hz, J₂=5.5 Hz), 3.09-3.13(m, 2H), 3.43(t, 1H, J=7.3 Hz), 3.68(s, 3H), 3.82(d, 1H, J=10.7 Hz), 4.34(s, 1H), 6.96 (d, 1H, J=8.9 Hz), 7.17(d, 1H, J=8.1 Hz), 7.20 (d, 1H, J=12 Hz), 7.26-7.32 (m, 5H), 7.40 (t, 1H, J=7.6 Hz), 7.50 (d, 1H, J=7.7 Hz), 7.60 (d, 1H, J=8.0 Hz), 7.70 (s, 1H); Mass Spectrum: m/e=527 (M+1, ³⁵Cl ⁷⁹Br), 529 (M+1, ³⁷Cl⁷⁹Br/³⁵Cl ⁸¹Br), 531 (M+1, ³⁷Cl ⁸¹Br).

### Step 8 3-[(S)-{3-[(1S)-1-(3-bromo-5-fluorophenyl)-2-hydroxy-2-methylpropyl]azetidin-1-yl}(4-chlorophenyl)methyl]benzonitrile

A portion of 7.84 g (31.80 mmol) of cerium (III) chloride (anhydrous) was stirred for 30 min under N₂ at rt. Then 120 mL of THF (anhydrous) was added, and the slurry was stirred for another 30 min at rt. Then the reaction mixture was cooled to −78° C. and a solution of 14.9 mL (23.80 mmol) methyl lithium (1.6M in ether) was added drop wise. After addition was complete, the mixture was stirred for 30 min at −78° C. Then a solution of 6.0g (11.36 mmol) of methyl (2S)-(3-bromo-5-fluorophenyl){1-[(S)-(4-chlorophenyl)(3-cyanophenyl)methyl]azetidin-3-yl}acetate in 30 mL of THF was added, and the reaction mixture was stirred for 1.5 h at −78° C. The reaction was quenched by drop wise addition of 20 mL water and warmed slowly to rt. The pH was adjusted to 7-8 with aqueous NaHCO₃. The solid residue was washed with CH₂Cl₂ (300 mLx4). The combined organic layer was dried over Na₂S0₄ and concentrated. The residue was purified by silica gel chromatography with hexane/ethyl acetate to afford the title compound as a white solid; ¹H-NMR(CDCl₃) δ 1.10 (s, 3H), 1.15 (s, 3H), 2.28(t, 1H, J=7.8 Hz), 2.71(d, 1H, J₁=11 Hz), 2.84 (t, 1H, J=7.5 Hz), 3.09-3.18(m, 2H), 3.60 (m, 1H), 4.25(s, 1H), 6.86 (d, 1H, J=9.4 Hz), 7.10-7.13(m, 2H), 7.28-7.34 (m, 4H), 7.36 (t, 1H, J=7.8 Hz), 7.46 (d, 1H, J=7.5 Hz), 7.57 (d, 1H, J=7.5 Hz), 7.67 (s, 1H); Mass Spectrum: m/e=527, (M+1, ³⁵Cl ⁷⁹Br), 529 (M+1, ³⁷Cl⁷⁹Br/³⁵Cl ⁸¹Br), 531 (M+1, ³⁷Cl ⁸¹Br).

### Step 9 3-[(S)-{3-[(1S)-1-(3-bromo-5-fluorophenyl)-2-fluoro-2-methylpropyl]azetidin-1-yl}(4-chlorophenyl)methyl]benzonitrile

A mixture of 6.90 g (13.07 mmol) of 3-[(S)-{3-[(1s)-1-(3-bromo-5-fluorophenyl)-2-hydroxy-2-methylpropyl]azetidin-1-yl}(4-chlorophenyl)methyl]benzonitrile, 50 mL of hydrogen fluoride pyridine (HF 70%), and 60 mL of 1,2-dichloroethane was stirred at 40-42° C. for 16 h. Then the reaction mixture was poured slowly to a mixture of 250 mL of water, 74 g of NaOH, 300 mL of aqueous NaHCO₃, 300 g of ice and 500 mL of CH₂Cl₂ with rapid stirring. The pH of the mixture was adjusted to 7-8, and the mixture was filtered to remove solids. The aqueous layer was extracted with three 300 mL portions of CH₂Cl₂. The combined organic layer was dried over Na₂S0₄ and concentrated. The residue was purified by silica gel chromatography with hexane/ethyl acetate to afford the title compound as a white solid; ¹H-NMR(CDCl₃) δ 1.25 (t, 6H, J=11.8 Hz), 2.32(t, 1H, J=7.5 Hz), 2.83-2.89(m, 2H), 3.09-3.17(m, 2H), 3.59 (m, 1H), 4.26(s, 1H), 6.85 (d, 1H, J=9.1 Hz), 7.10-7.14(m, 2H), 7.28-7.33 (m, 4H), 7.36 (t, 1H, J=7.2 Hz), 7.46 (d, 1H, J=7.8 Hz), 7.56 (d, 1H, J=7.7 Hz), 7.67 (s, 1H); Mass Spectrum: m/e=529, (M+1, ³⁵Cl ⁷⁹Br), 531 (M+1, ³⁷Cl⁷⁹Br/³⁵Cl ⁸¹Br), 533 (M+1, ³⁷Cl ⁸¹Br).

### Step 10 3-[(S)-{3-[(1S)-1-(3-bromo-5-fluorophenyl)-2-fluoro-2-methylpropyl]azetidin-1-yl}(4-chlorophenyl)methyl]benzoic acid

A mixture of 5.52 g (10.4 mmol) of 3-[(S)-{3-[(1S)-1-(3-bromo-5-fluorophenyl)-2-fluoro-2-methylpropyl]azetidin-1-yl}(4-chlorophenyl)methyl]benzonitrile, 65 mL of EtOH, and 40 mL of 5N NaOH was heated to reflux for 4 h (with monitoring by HPLC). Then the pH solution was adjusted to 4-5 with 12N HCl and the solution was concentrated to remove organic solvents. The residue was dissolved in 200 mL of CH₂Cl₂ and filtered to remove undissolved solid. The solid residue was triturated with two 200 mL portions of CH₂Cl₂ and the combined organic layers were concentrated to the title compound as white solid; Mass Spectrum: m/e=548, (M+1, ³⁵Cl ⁷⁹Br), 550 (M+1, ³⁷Cl⁷⁹Br/³⁵Cl ⁸¹Br), 552 (M+1, 37Cl ⁸¹Br).

### Step 11 Ethyl 3-[(S)-{3-[(1S)-1-(3-bromo-5-fluorophenyl)-2-fluoro-2-methylpropyl]azetidin-1-yl}(4-chlorophenyl)methyl]benzoate

To a mixture of 5.72 g (10.4 mmol) of 3-[(S)-{3-[(1S)-1-(3-bromo-5-fluorophenyl)-2-methylpropyl]azetidin-1-yl}(4-chlorophenyl)methyl]benzoic acid in 230 mL of EtOH, was added a solution of 25 mL of 4N HCl in dioxane. After 7.5 h at reflux, the solution was cooled and concentrated to remove solvents. To the residue were added 150 mL of CH₂Cl₂ and 30 mL of H₂O and the pH was adjusted to 7-8 with aqueous NaHCO₃. The aqueous layer was extracted with three 100 mL portions of CH₂Cl₂and the combined organic layer was dried over Na₂SO₄ and concentrated. The residue was purified by silica gel chromatography with hexanes/ethyl acetate to afford the title compound as a white solid; ¹H-NMR(CDCl₃) δ 1.25 (d, 3H, J₁=22 Hz), 1.27 (d, 3H, J₁=22 Hz), 1.40(t, 3H, J=7.1 Hz), 2.34(t, 1H, J=7.5 Hz), 2,83-2.89(m, 2H), 3.10-3.15(m, 2H), 3.62 (m, 1H), 4.30(s, 1H), 4.38 (q, 2H, J₁=14.2 Hz, J₂=7.1 Hz), 6.85 (d, 1H, J=9.4 Hz), 7.10-7.11(m, 2H), 7.26-7.38 (m, 5H), 7.55 (d, 1H, J=7.8 Hz), 7.86 (d, 1H, J=7.7 Hz), 8.05 (s, 1H); Mass Spectrum: m/e=576, (M+1, ³⁵Cl ⁷⁹Br), 578 (M+1, ³⁷Cl⁷⁹Br/³⁵Cl ⁸¹Br), 580 (M+1, ³⁷Cl ⁸¹Br).

### Step 12 Ethyl 3-((S)-(4-chlorophenyl){3-[(1S)-1-(3-cyano-5-fluorophenyl)-2-fluoro-2-methyl-propyl]azetidin-1-yl}methyl)benzoate

A mixture of 5.64 g (9.77 mmol) of ethyl 3-[(S)-{3-[(1S)-1-(3-bromo-5-fluorophenyl)-2-fluoro-2-methylpropyl]azetidin-1-yl}(4-chlorophenyl)methyl]benzoate, 700 mg (5.96 mmol) of zinc cyanide, 179 mg (0. 195 mmol) of tris(dibenzylideneacetone)dipalladium, and 270 mg (0.489 mmol) of DPPF in 99 mL of DMF and 1 mL of water was degassed with N₂ for 1 h at rt. Then it was stirred at 125° C. After 12 h, the reaction mixture was concentrated to remove solvents. The residue was poured into 200 mL of CH₂Cl₂ and 50 mL of aqueous NaHCO3 (pH=7-8). The aqueous layer was extracted with CH₂Cl₂and the combined organic layer was dried over Na₂S0₄ and concentrated. The residue was purified by silica gel chromatography with hexanes/ethyl acetate to afford the title compound as a white solid; ¹H-NMR(CDCl₃) δ 1.23 (d, 3H, J₁=21.7 Hz), 1.28 (d, 3H, J₁=21.7 Hz), 1.40(t, 3H, J=7.1 Hz), 2.29(t, 1H, J=7.5 Hz), 2.85-2.95(m, 2H), 3.05(m, 1H), 3.18(m, 1H), 3.64 (t, 1H, J=6.7 Hz), 4.29(s, 1H), 4.37 (q, 2H, J₁=14.2 Hz, J₂=7.1 Hz), 7.16 (d, 1H, J=9.3 Hz), 7.23 (d, 1H, J=7.8 Hz), 7.27-7.38(m, 6H), 7.55 (d, 1H, J=7.8 Hz), 7.86 (d, 1H, J=7.7 Hz), 8.0 (s, 1H); Mass Spectrum: m/e=523, (M+1, ³⁵Cl), 525 (M+1, ³⁷Cl).

### Step 13 3-((S)-(4-chlorophenyl){3-[(1S)-1-(3-cyano-5-fluorophenyl)-2-fluoro-2-methylpropyl]azetidin-1-yl}methyl)benzohydrazide

A mixture of 3.06 g (5.85 mmol) of ethyl 3-((S)-(4-chlorophenyl){3-[(1S)-1-(3-cyano-5-fluorophenyl)-2-fluoro-2-methylpropyl]azetidin-1-yl}methyl)benzoate, 13 mL (16.36 mmol) of hydrazine, and 85 mL of EtOH was heated to reflux. After 8 h, the solvents were removed to afford the title compound as a white solid.; ¹H-NMR(CDCl₃) δ 1.22 (d, 3H, J₁=21.7 Hz), 1.28 (d, 3H, J₁=21.7 Hz), 2.29(t, 1H, J=7.5 Hz), 2.85-2.94(m, 2H), 3.07(m, 1H), 3.15(m, 1H), 3.62 (t, 1H, J=6.6 Hz), 4.29(s, 1H), 7.16 (d, 1H, J=9.1 Hz), 7.22 (d, 1H, J=7.5 Hz), 7.25-7.36(m, 6H), 7.50-7.54 (m, 2H), 7.77 (s, 1H); Mass Spectrum: m/e=509, (M+1, ³⁵Cl), 511 (M+1, ³⁷Cl).

### Step 14 3-[(1S)-1-(1-{(S)-(4-chlorophenyl)[3-(5-oxo-4,5-dihydro-1,3,4- oxadiazol-2-yl)phenyl]methyl}azetidin-3-yl)-2-fluoro-2-methylpropyl]-5-fluorobenzonitrile

To a solution of 3.0 g (5.85 mmol) of 3-((S)-(4-chlorophenyl){3-[(1S)-1-(3-cyano-5-fluorophenyl)-2-fluoro-2-methylpropyl]azetidin-1-yl}methyl)benzohydrazide in 100 mL of CH₂Cl₂ was added 3.73 mL (7.1 mmol) of phosgene solution (20% in toluene). The solution was stirred for 1.5 h at 0° C. The solution was concentrated, and to the residue was added 6 mL of 2N NH₃ in MeOH, and the mixture was concentrated again. The residue was purified by silica gel chromatography with CH₂Cl₂/acetone to afford the title compound as a white solid; ¹H-NMR(CDCl₃) δ 1.26 (d, 3H, J₁=22.1 Hz), 1.30 (d, 3H, J₁=21.7 Hz), 2.39(t, 1H, J=8.0 Hz), 2.93-3.0(m, 2H), 3.12(t, 1H, J=7.3 Hz), 3.33(m, 1H), 3.69 (t, 1H, J=6.8 Hz), 4.35(s, 1H), 7.20 (d, 1H, J=9.7 Hz), 7.23 (d, 1H, J=7.3 Hz), 7.29-7.40(m, 7H), 7.48 (d, 1H, J=7.8 Hz), 7.68 (d, 1H, J=7.8 Hz), 7.97 (s, 1H); Mass Spectrum: m/e=535, (M+1, ³⁵Cl), 537 (M+1, ³⁷Cl).

### Example 2

### Solubility of 3-[(1S)-1-(1-{(S)-(4-chlorophenyl)[3-(5-oxo-4,5-dihydro-1,3,4-oxadiazol-2-yl)phenyl]methyl}azetidin-3-yl)-2-fluoro-2-methylpropyl]-5-fluorobenzonitrile (Compound I)

The solubility of Compound I was determined in various vehicles as shown in Table 1. Solubility of Compound 1 was determined using a high throughput screening method.

TABLE 1

| | Compound I free base | |
|---|---|---|
| | Vehicle at ambient | |
| Vehicle | temperature | |
| 0.01N HCl/34 mMNaCl | 0.007 | mg/g |
| Water | 0.001 | mg/g |
| MIGLYOL 812 | 86 | mg/g |
| IMWITOR 742 | 318 | mg/g |
| IMWITOR:TWEEN (1:1) | >240 | mg/g |
| POLYSORBATE 80 (aqueous solution) | <40 | μg/ml |
| Sodium lauryl sulfate (aqueous solution) | <40 | μg/ml |
| CREMOPHOR EL (aqueous solution) | <40 | μg/ml |
| Vitamin E TPGS (aqueous solution) | <40 | μg/ml |
| Methyl Cellulose (aqueous solution) | <40 | μg/ml |
| Methyl Cellulose with SDS (aqueous solution) | <40 | μg/ml |
| Simulated gastric fluids (fasted and fed) | <40 | μg/ml |

The above solubilities were determined by adding Compound I to the test system and stirred. Following an equilibrium time, the material in the solution phase is assayed by HPLC to determine solubility.

### Example 3

### Formulation of Liquid-Filled Capsules

Formulations containing Compound I that were used to fill capsules are shown in Table 2.

TABLE 2

| Ingredient | Formulation A | Formulation B | Formulation C |
|---|---|---|---|
| Compound I | 1 mg | 5 mg | 25 mg |
| MIGLYOL 812 | 498.5 mg | 494.95 mg | 474.95 mg |
| Butylated | 0.05 mg | 0.05 mg | 0.05 mg |
| hydroxyaniole Flakes | | | |
| (BHA) | | | |

Compound I, MIGLYOL and BHA were combined in an appropriate vessel and the resulting solution used to fill 96 Size 0 (HG) capsules which were LEMS or band sealed.

### Example 4

### Stability of Formulations

The stabilities of various formulations are shown in Tables 3-5. Degradation of Compound I was monitored through HPLC assay. Relative % Initial compares the percent of degradation of the product in the formulation to the label claim. Relative Area % (based on area percent by HPLC assay response), 270 nm shows the percentage of the active peak of Compound I relative to all other peaks in the chromatogram.

TABLE 3
Formulation: 2.5 mg of Compound I/g IMWITOR-TWEEN 80 (1:1)

| | Relative % Initial | | | | Relative Area %, 270 nm | | | |
|---|---|---|---|---|---|---|---|---|
| | 0.05 | | 0.05 | | 0.05 | 0.05 | | 0.05 |
| | mg | 0.05 mg | mg | 0.05 mg | mg | mg | 0.05 mg | mg |
| −20° C. | 100 | 100 | 100 | 100 | 100 | 100 | 100 | 100 |
| 40° C./ | 80.3 | 71.4 | 58.2 | 51.1 | 88.5 | 80.8 | 71.5 | 64.6 |
| amb RH | | | | | | | | |

TABLE 4
Formulation: 2.2 mg of Compound I/g IMWITOR-TWEEN
80 (1:1)/0.02% BHA

| | Rel % Initial | | | | Relative Area %, 270 nm | | | |
|---|---|---|---|---|---|---|---|---|
| | 0.05 | | 0.05 | | 0.05 | | 0.05 | 0.05 |
| | mg | 0.05 mg | mg | 0.05 mg | mg | 0.05 mg | mg | mg |
| −20° C. | 100 | 100 | 100 | 100 | 100 | 100 | 100 | 100 |
| 40° C./ | 96.5 | 93.3 | 94.2 | 91.5 | 97.7 | 99.3 | 98.4 | 97.7 |
| amb RH | | | | | | | | |

TABLE 5
Formulation: 2.3 mg of Compound I/g IMWITOR

| | Rel % Initial | | | | Relative Area %, 270 nm | | | |
|---|---|---|---|---|---|---|---|---|
| | 0.05 | | 0.05 | | 0.05 | 0.05 | | 0.05 |
| | mg | 0.05 mg | mg | 0.05 mg | mg | mg | 0.05 mg | mg |
| −20° C. | 100 | 100 | 100 | 100 | 100 | 100 | 100 | 100 |
| 40° C./ | 101.4 | 99.1 | 98.5 | 99.1 | 100.7 | 100.3 | 99.7 | 99.7 |
| amb RH | | | | | | | | |

The IMWITOR:TWEEN formulations showed evidence of oxidation that was noted on stability. The formulations in just IMWITOR showed greater stability.

### Example 5

### Mean Pharmacokinetics Parameters after Oral Administration of Compound I to Rhesus Monkeys at a Dose of 0.14 mpk [n=3, mean±SE(SD)]

In-vitro evaluation of formulations was conducted in rhesus monkeys. The results are shown in Table 6.

TABLE 6

| | MIGLYOL LFC | IMWITOR LFC |
|---|---|---|
| AUC 0-24 hr | 91.8 ± 6.76 (11.7) | 69.2 ± 9.91 (17.2) |
| (nM * hr) | | |
| Cmax | 9.26 ± 1.10 (1.90) | 10.7 ± 2.78 (4.82) |
| (nM) | | |
| Tmax | 91.8 ± 6.76 (11.7) | 91.8 ± 6.76 (11.7) |
| (hr) | | |
| AUC0-24 hr/AUC0-24 | 100 | 75 (±21) |
| MIGLYOL | | |

Fasted male rhesus monkeys (New Iberia, LA) were used for the studies. Following an overnight fast, animals received capsules via oral gavage immediately followed by 3.5 ml/kg of water. The animals had free access to water except between dosing and 1 hour after dosing. A 1-ml blood sample was drawn via venipuncture and using a 21 g butterfly needle inserted into the saphenous vein at pre-dose and 0.25, 0.5, 1, 2, 4, 6, 8 and 24 hours after dosing The plasma was separated by centrifugation (10 minutes at 2500 rpm) and kept frozen at −70C until analysis.

### Example 6

### Tablet Formulations

Table formulations are shown in Table 7.

TABLE 7

| | 0.1 mg | 0.25 mg | 0.5 mg |
|---|---|---|---|
| | Strength Tablet | Strength Tablet | Strength Tablet |
| Compound I | 0.10 | 0.25 | 0.5 |
| BHA | 0.02 | 0.02 | 0.02 |
| Microcrystalline | 20.0 | 20.0 | 20.0 |
| cellulose | | | |
| Lactose monohydrate | 73.88 | 73.73 | 73.48 |
| Croscarmellose | 3 | 3 | 3 |
| Sodium | | | |
| Hydroxypropyl | 2 | 2 | 2 |
| cellulose | | | |
| Magnesium Stearate | 1 | 1 | 1 |
| Water USP Purified | Removed | Removed | Removed |
| | during drying | during drying | during drying |
| Alcohol, Ethyl SD3A | Removed | Removed | Removed |
| | during drying | during drying | during drying |

Compound I, microcrystalline cellulose, lactose, hydroxypropyl cellulose, croscarmellose sodium and butyl hydroxyanisole (BHA) were combined and granulated. The granulation solvent is a mixture of ethanol and water. The resulting granules are lubricated with magnesium stearate and compressed.

While the invention has been described and illustrated with reference to certain particular embodiments thereof, those skilled in the art will appreciate that various changes, modifications and substitutions can be made therein without departing from the spirit and scope of the invention. For example, solvents other than the particular solvents as set forth herein above may be useful in the chemical syntheses described herein. It is intended, therefore, that the invention be defined by the scope of the claims which follow and that such claims be interpreted as broadly as is reasonable.

What is claimed is:

1. A pharmaceutical composition comprising 3-[(1S)-1-(1-{(S)-(4-chlorophenyl)[3-(5-oxo-4,5-dihydro-1,3,4-oxadiazol-2-yl)phenyl]methyl}azetidin-3-yl)-2-fluoro-2-methylpropyl]-5-fluorobenzonitrile, a triglyceride and at least one additional pharmaceutically acceptable excipient.

2. The pharmaceutical composition of claim 1 wherein the triglyceride is caprylic/capric glyceride, fractionated coconut oils or a combination thereof.

3. The pharmaceutical composition of claim 1, wherein an additional pharmaceutically acceptable excipient is at least one antioxidant.

4. The pharmaceutical composition of claim 3, wherein the antioxidant is BHA, BHT, PG, Vitamin E or AP or a combination thereof.

5. The pharmaceutical composition of claim 1, wherein an additional pharmaceutically acceptable excipient is at least one surfactant.

6. The pharmaceutical composition of claim 5, wherein the surfactant is a polyoxyethylene sorbitan fatty acid ester.

7. The pharmaceutical composition of claim 1, wherein the pharmaceutical composition is formulated for oral administration.

8. The pharmaceutical composition of claim 1, wherein the pharmaceutical composition is a capsule.

9. A composition for the treatment of obesity comprising 3-[(1S)-1-(1-{(S)-(4-chlorophenyl)[3-(5-oxo-4,5-dihydro-1,3,4-oxadiazol-2-yl)phenyl]methyl}azetidin-3-yl)-2-fluoro-2-methylpropyl]-5-fluorobenzonitrile, a triglyceride and at least one additional pharmaceutically acceptable excipient.

10. A composition for the treatment of Alzheimer's Disease comprising 3-[(1S)-1-(1-{(S)-(4-chlorophenyl)[3-(5-oxo-4,5-dihydro-1,3,4-oxadiazol-2-yl)phenyl]methyl}azetidin-3-yl)-2-fluoro-2-methylpropyl]-5-fluorobenzonitrile, a triglyceride and at least one additional pharmaceutically acceptable excipient.

11. The composition of claims 9, wherein the triglyceride is caprylic/capric glyceride, fractionated coconut oils or a combination thereof.

12. The composition of claims 9, wherein an additional pharmaceutically acceptable excipient is at least one antioxidant.

13. The composition of claim 12, wherein the antioxidant is BHA, BHT, PG, Vitamin E or AP or a combination thereof.

14. The composition of claims 9, wherein an additional pharmaceutically acceptable excipient is at least one surfactant.

15. The composition of claim 14, wherein the surfactant is a polyoxyethylene sorbitan fatty acid ester.

16. The composition of claims 9, wherein the pharmaceutical composition is formulated for oral administration.

17. The composition of claims 9, wherein the pharmaceutical composition is a capsule.
